SEQUENCE LISTING (8 sequences)

SEQ ID NO: 1 - protein, 59 aa, synthetic construct
MSFVDHPPDWLEEVGEGLREFLGLEAGPPKPKPNQQHQDQARGLVLPGYNYLGPGNGLD

SEQ ID NO: 2 - protein, 60 aa, synthetic construct
MAADGYLPDWLEDNLSEGIREWWDLKPGAPKPKANQQKQDDGRGLVLPGYKYLGPFNGLD

SEQ ID NO: 3 - protein, 60 aa, synthetic construct
MAADGYLPDWLEDTLSEGIRQWWKLKPGPPPPKPAERHKDDSRGLVLPGYKYLGPFNGLD

SEQ ID NO: 4 - protein, 60 aa, synthetic construct
MAADGYLPDWLEDNLSEGIREWWALKPGVPQPKANQQHQDNRRGLVLPGYKYLGPGNGLD

SEQ ID NO: 5 - protein, 60 aa, synthetic construct
MAADGYLPDWLEDNLSEGIREWWALKPGAPQPKANQQHQDNARGLVLPGYKYLGPGNGLD

SEQ ID NO: 6 - protein, 60 aa, synthetic construct
MAADGYLPDWLEDNLSEGIREWWALKPGAPKPKANQQKQDDGRGLVLPGYKYLGPFNGLD

SEQ ID NO: 7 - protein, 60 aa, synthetic construct
MAADGYLPDWLEDNLSEGIREWWDLKPGAPKPKANQQKQDNGRGLVLPGYKYLGPFNGLD

SEQ ID NO: 8 - protein, 59 aa, synthetic construct
MTDGYLPDWLEDNLSEGVREWWALQPGAPKPKANQQHQDNARGLVLPGYKYLGPGNGLD

(19) United States
(12) Patent Application Publication
(10) Pub. No.: US 2025/0145969 A1
(43) Pub. Date: May 8, 2025

(54) Deamidation Depleted Adeno-Associated Virus Product

(71) Applicant: Oxford Biomedica (US) LLC, Bedford, MA (US)

(72) Inventors: Alex Meola, Bedford, MA (US); Alice Aguiar, Bedford, MA (US); Carlos Chong, Bedford, MA (US); Zaid Junaid, Bedford, MA (US); Thomas Thiers, Bedford, MA (US); Eli Wiberg, Bedford, MA (US); Ify Iwuchukwu, Bedford, MA (US); Guang Yang, Bedford, MA (US)

(21) Appl. No.: 18/940,528

(22) Filed: Nov. 7, 2024

Related U.S. Application Data
(60) Provisional application No. 63/597,038, filed on Nov. 8, 2023.
(60) Provisional application No. 63/625,575, filed on Jan. 26, 2024.
(60) Provisional application No. 63/636,594, filed on Apr. 19, 2024.

(51) Int. Cl. C12N 7/00; A61K 48/00; B01D 15/32; B01D 15/36; C07K 14/005; C12N 15/86

(52) U.S. Cl. CPC C12N 7/00; A61K 48/0041; B01D 15/327; B01D 15/363; C07K 14/005; C12N 15/86; C12N 2750/14122; C12N 2750/14143; C12N 2750/14151

(57) ABSTRACT

Provided is an adenovirus-associated virus (AAV) product containing fewer non-functional capsids, such as deamidated empty AAV capsids, deamidated AAV capsids comprising a portion of the transgene, and deamidated full capsids, than AAV products currently in use. The AAV product contains a higher ratio of functional capsids to non-functional capsids (deamidated, hydrophobic capsids), compared to a control AAV product purified solely by ultracentrifugation, by anion-exchange chromatography (AEX) without a fatty acid mobile phase. Methods of making and using the AAV product are also provided, including separating functional capsids from non-functional capsids with anion-exchange chromatography (with fatty acid mobile phase), HIC, or chromatography with a mixed mode resin. Such methods also include maintaining transfected cells in a bioreactor about two days post-transfection, optionally at about 30-38° C., and subsequently separating the functional capsids from the non-functional capsids.

### CROSS-REFERENCE TO RELATED APPLICATIONS

This application claims the benefit of priority of U.S. Provisional Patent Application No. 63/597,038, filed on Nov. 8, 2023; U.S. Provisional Patent Application No. 63/625,575, filed on Jan. 26, 2024; and U.S. Provisional Patent Application No. 63/636,594, filed on Apr. 19, 2024.

### SEQUENCE LISTING

The instant application contains a Sequence Listing which has been submitted electronically in XML format and is hereby incorporated by reference in its entirety. Said XML copy, created on Oct. 17, 2024, is named OBM_027_US1_SL.xml and is 8,427 bytes in size.

### BACKGROUND

Adeno-associated viral (AAV) vectors are commonly used for nucleic acid delivery into cells. Successes in AAV-mediated gene replacement, gene silencing, and gene editing make AAV a desirable therapeutic vector, with AAV-based therapeutics gaining regulatory approval in Europe and the United States. However, existing AAV purification systems often fail to remove impurities from AAV products and frequently produce insufficient quantities of vector genomes for robust gene therapy applications. Accordingly, improved AAV products and AAV product purification systems are needed.

### SUMMARY

This disclosure relates to an AAV product that contains fewer non-functional capsids, such as deamidated empty capsids, deamidated capsids containing only a partial transgene, and deamidated full capsids than AAV products concurrently in use. The AAV product of the present invention contains fewer deamidated, hydrophobic capsids, which represent non-functional capsids. Because the improved AAV product has fewer non-functional capsids, the product results in better transgene expression with a given concentration of capsids. Furthermore, the improved product results in less immunoreactivity in a subject administered the AAV product because, by reducing the number of empty capsids or capsids with a partial transgene, the capsid protein amount in a single effective dose is reduced.

Thus, provided herein is an AAV product comprising functional capsids containing a transgene and fewer deamidated AAV capsids as compared to a control AAV product. For example, in some cases the AAV product comprises (a) functional capsids, wherein the functional capsids contain a transgene; and (b) fewer deamidated capsids than a control AAV product purified solely by ultracentrifugation or by AEX without a fatty acid mobile phase. In some embodiments, the AAV product comprises less than 10%, less than 5%, or less than 1% capsids having a deamidated N57 on VP1. In some embodiments, the AAV product comprises fewer N57 deamidated capsids than the control AAV product purified solely by AEX without a fatty acid mobile phase.

Further provided is an adenovirus-associated virus (AAV) product comprising functional capsids, wherein the functional capsids contain a functional transgene and wherein the product is less hydrophobic than a control AAV product purified solely by ultracentrifugation or by AEX without a fatty acid mobile phase. The adenovirus-associated virus (AAV) product comprises more functional capsids than a control AAV product purified solely by ultracentrifugation or by AEX without a fatty acid mobile phase,

In some embodiments, the AAV product comprises less than 10% non-functional capsids. Given the reduction in non-functional capsids, the AAV product has a higher ratio of functional capsids to non-functional capsids, and a given concentration of capsids has greater transgene expression in a host cell than a control AAV product of the same concentration purified solely by ultracentrifugation or AEX without a fatty acid mobile phase. Thus, also provided is an AAV product comprising AAV capsids containing a transgene, wherein a concentration of the AAV capsids has greater transgene expression in a host cell than the same concentration of control AAV capsids purified solely by ultracentrifugation or AEX without fatty acid mobile phase.

In some embodiments, the AAV product has a lower ratio of VP1 to VP2 than a control AAV product purified solely by ultracentrifugation or AEX without fatty acid mobile phase. Thus, provided herein is an AAV product comprising AAV capsids containing a transgene, wherein the AAV product has a lower ratio of VP1 to VP2 than a control AAV product purified solely by ultracentrifugation or AEX without fatty acid mobile phase.

Also provided is an AAV product consisting essentially of functional capsids containing a functional transgene and a buffer. In some embodiments, the functional capsids are separated from a population of AAV capsids comprising functional capsids and deamidated capsids to produce the AAV product. In some embodiments, the AAV product is relatively devoid of capsids with VP1 having a deamidated N57.

Also provided is a method of removing at least a sub-population of non-functional capsids from an adenovirus-associated virus (AAV) product comprising separating functional capsids containing a transgene from a subpopulation of non-functional capsids based on hydrophobicity. Also provided is an AAV product produced by any of the methods described herein.

In some methods, separating the functional capsids from the non-functional capsids is further based on charge. In some methods, the removed non-functional capsids comprise deamidated capsids. In some methods, the removed deamidated capsids comprise VP1 with deamidated N57.

Optionally, separating functional capsids containing the functional transgene from the subpopulation of non-functional capsids comprises a hydrophobic interaction chromatography (HIC) step, an AEX step with a fatty acid mobile phase, or a mixed-mode AEX step. When a HIC step is used, an AEX step (e.g., without a fatty acid mobile phase) can also be performed. In such a case, the HIC step optionally precedes the AEX step. When the separation step comprises an AEX step with a fatty acid mobile phase, the fatty acid mobile phase optionally comprises octanoic acid (also known as octanoate, caprylate, or caprylic acid) or heptanoic acid (also known as heptanoate and enanthic acid). When the separation comprises a mixed-mode resin chromatography step, the mixed mode resin is optionally a hydrophobic anion exchange resin.

Also provided is a method for reducing the number of non-functional capsids by maintaining the time that the cells are retained in a bioreactor post-transfection to about two days, optionally while maintaining a temperature in the reactor of about 30-38° C., and separating functional capsids containing the functional transgene from the subpopulation of non-functional capsids using AEX (with or without a fatty acid mobile phase and with or without a mixed-mode resin) or HIC.

Further provided is a method of treating a subject in need of a protein produced by a transgene comprising administering to the subject any of the AAV products described herein.

### BRIEF DESCRIPTION OF THE DRAWINGS

The figures described below are intended to illustrate certain embodiments and/or features of the compositions and methods and to supplement any description(s) of the compositions and methods. The figures do not limit the scope of the compositions and methods, unless the written description expressly indicates that such is the case.

FIG. 1A is a schematic showing empty capsids, partial capsids, and full capsids with an intact target nucleic acid within the capsid. Charge of the capsids theoretically increases with the presence and amount of nucleic acid present inside the capsid. FIG. 1B is an example of an AEX linear gradient showing the leading empty capsids followed by the product containing functional transgenes, and the tailing non-product peak consisting of non-functional capsids (i.e., deamidated capsids).

FIG. 2A shows that deamidation of VP1 imparts a negative charge and that the N terminus of VP1 is hydrophobic across AAV serotypes. Deamidation of N57 of VP1 results in a loss of potency and a drop in transgene expression, presumably caused by VP1 extrusion on the capsid surface. FIG. 2B shows the percentage of deamidation for residues N329, N57, Q456, and N452 of VP1 at 25° C. from 0-4 weeks (upper panel) and at 40° C. from 0-10 days (lower panel). Also shown in each graph is the relative potency of the AAV product, which decreases as deamidation increases over time.

FIG. 3 shows a chromatogram of anion exchange (AEX) chromatography comparing the chromatographic performance of control Poros HQ50 resin (Thermo Scientific, Waltham, MA) to Nuvia aPrime 4A resin for the enrichment of DNA containing capsids. AEX separated the capsids into two distinct peaks (separated by a vertical dashed black line): Pool 1 product peak which comprises the mostly full capsids (left), and Pool 2 non-product peak which comprises the mostly empty capsids (right). The Nuvia aPrime 4A resin provided better chromatographic resolution between Pool 1 and Pool 2, in comparison to the control Poros HQ50 resin.

FIG. 4 shows the effect on chromatographic performance when the mobile phase of POROS HQ50 AEX chromatography is modified to incorporate the fatty acid heptanoate. The tested mobile phases include AEX chromatography without fatty acid mobile phase; AEX chromatography mobile phase containing sodium heptanoate with ammonium acetate; and AEX chromatography mobile phase containing sodium heptanoate with glycine. The AEX chromatography mobile phase containing heptanoate increased chromatographic resolution between Pool 1 and Pool 2, as compared to control.

FIG. 5 is a representative chromatograph of a hydrophobic interaction chromatography (HIC) run, as described in the Examples.

FIG. 6 is a representative chromatograph showing the gradient elution of the HIC run of FIG. 5.

FIG. 7 is a graph showing the vector genome (VG) and capsid yield data based on affinity (AF) product titers normalized to mass balance. VG and capsid titers for the flow through and wash (FTW) fraction were below the limit of quantification and are not shown here. HIC Pool 1 contained most of the loaded material, comprising about 76% of capsid yield and 76% VG yield. The ratio of the VG titer per capsid titer was 19% for HIC Pool 1 and 22% for HIC Pool 2, suggesting this step did not enrich for DNA containing capsid.

FIG. 8 shows the analytical ultracentrifuge profiles of HIC Pool 1 and HIC Pool 2.

FIG. 9 is a chromatogram obtained for the AEX polishing step using buffer exchanged HIC Pool 1.

FIG. 10 is a chromatogram obtained for the AEX polishing step using buffer exchanged HIC Pool 2.

FIG. 11 is an overlay of the chromatographs for the two AEX runs (FIG. 9 and FIG. 10).

FIG. 12 shows a representative AEX chromatogram with gradient elution using affinity (AF) product material.

FIG. 13 shows VG and capsid yield data of the AEX chromatography step with BE HIC Pool 1. Yields were calculated based on the titers of the BE HIC pools (because of the variability in the AEX load titers) and normalized to mass balance.

FIG. 14 shows VG and capsid yield data of the AEX chromatography step with BE HIC Pool 2. Yields were calculated based on the titers of the BE HIC pools (because of the variability in the AEX load titers) and normalized to mass balance.

FIG. 15 shows the AUC profiles of AEX Pool 1 and AEX Pool 2 fractions of the AEX chromatography step using BE HIC Pool 1.

FIG. 16 is a bar graph showing the percent abundance of amino acid deamidation at various residues in VP1 that correspond to each chromatographic peak from the HIC chromatography experiments (S1: Pool 1 product peak and S2: Pool 2 non-product peak), AEX chromatography of BE HIC Pool 1 (S3: product peak 1 and S4: non-product peak 2), and AEX chromatography of BE HIC Pool 2 (S5: non-product peak 2). The S4 and S5 peaks (HIC Pool 1-AEX Pool 2 and HIC Pool 2-AEX Peak 2) display the highest abundance of deamidation at Asparagine 57 (N57) as compared to the other amino acid residues examined. The S4 and S5 peaks display the latest elution times, indicating the species comprising these peaks (mostly empty capsids) have a high net negative charge. These data are from the HIC and AEX runs described above for FIGS. 5-15.

FIG. 17A and FIG. 17B are bar graphs showing the percentage of VG (FIG. 17A) and capsids (FIG. 17B) with yield data normalized to mass balance from AEX chromatography. Adjusted yields were measured at 2- and 3-days AAV harvest (i.e., days after the day of transfection), where transfection conditions were either at 37° C. or 39° C. In FIG. 17A, VG titers showed a slight increase in loss to strip on Day 3 versus Day 2 for both temperatures indicating that the feed-stream is more negatively charged. In FIG. 17B, capsid titer showed a decrease in FTW loss on Day 3 versus Day 2 and an increase in capsid loss to strip on Day 3 versus Day 2 indicating that the feed-stream is more negatively charged.

FIG. 18 shows a bar graph for the percentage of AUCs following AEX chromatography with Day 2 and Day 3 AAV harvest at 37° C. or 39° C. From left to right, the percentage of product empty capsids is shown in medium blue, product partially filled capsids in light blue, product full capsids in dark blue, non-product empty capsids from strip in orange, non-product partially filled capsids from strip in green, and non-product full capsids from strip in red. A higher percentage of empty capsids in the product peak and full capsids in the non-product peak from the strip were observed on Day 3 versus Day 2 further supporting a more negatively charged feed-stream. The best AAV packaging was observed at 39° C. on Day 2.

FIG. 19A shows a bar graph for the percent relative potency in the product peak after AEX chromatography. A drop in potency was observed from Day 2 to Day 3 at both temperatures, suggesting that the capsids harvested at 37° C. on Day 2 have the highest potency. FIG. 19B also shows a bar graph for the percent deamidation at N57. An increase of N57 deamidation was observed from Day 2 to Day 3 at both temperatures for both affinity (AF) product and AEX product peaks. Notably, 39° C. on Day 3 showed the lowest percent relative potency and the highest percentage of N57 deamidation.

FIGS. 20A and 20B are bar graphs illustrating VG and capsid productivity, respectively, with harvest at Day 2 or Day 3 at different temperatures. For both VG and capsid, an increase in upstream productivity from Day 2 to Day 3 at both 37° C. and 39° C. was observed. However, a subsequent decrease in total VGs and capsids recovered through the process from Day 2 to Day 3 at both temperatures was also noted. Together, these data show that Day 2 at 37° C. is the best condition for total process VG and capsid productivity.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

### AAV Products

Typically, AAV products are produced in cell culture, for example, in a bioreactor, followed by purification of the AAV products from the cell culture by eliminating at least a portion of impurities (e.g., removal of host cell proteins, media components, and/or free nucleic acids from the cell culture) and empty capsids (i.e., capsids lacking a transgene, including empty capsids). As disclosed herein, production of non-functional capsids can be reduced by limiting the time the cells remain in the bioreactor after transfection and before the AAV products are isolated and purified. For example, when the cells remain in the bioreactor for two days or less, fewer non-functional capsids are present in the population of unseparated capsids.

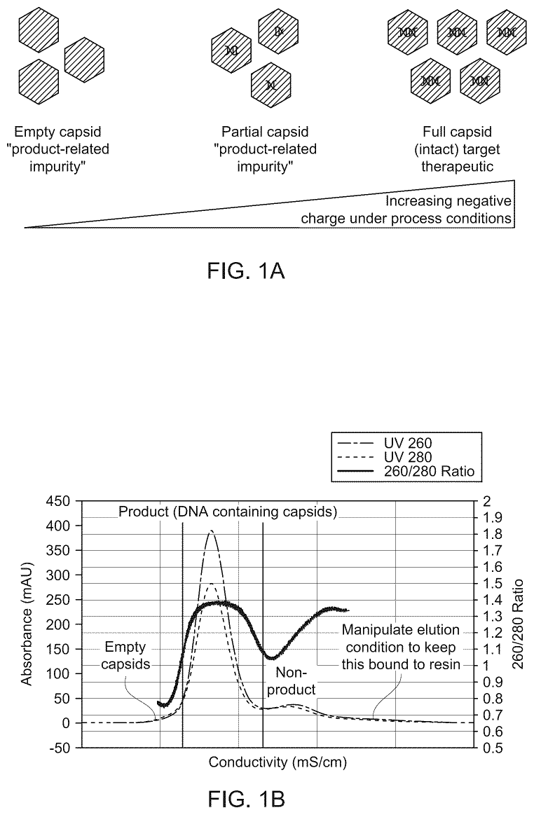

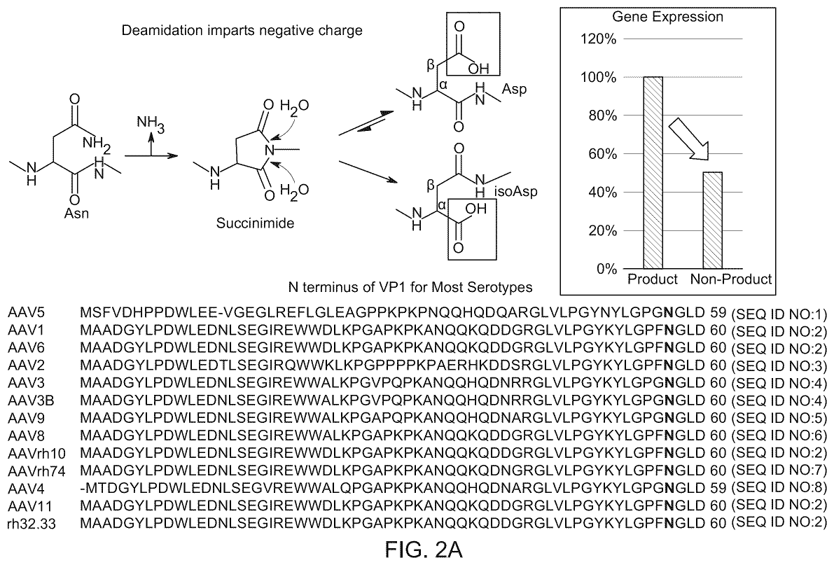

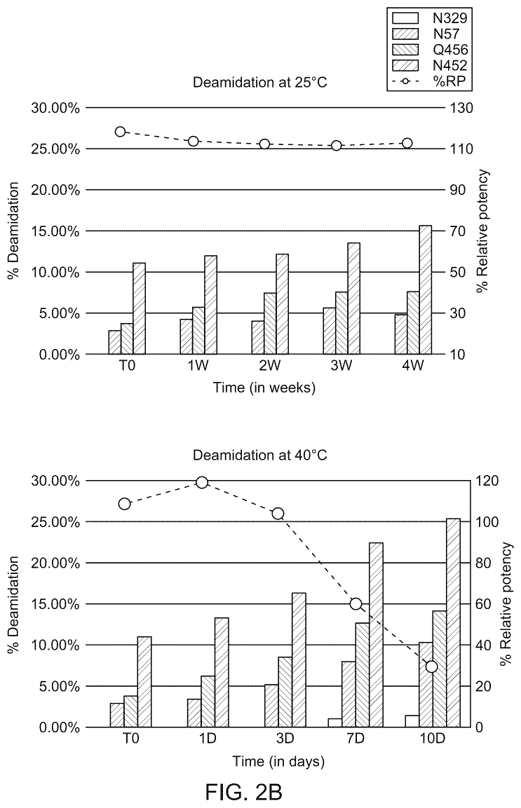

Purification further provides an opportunity to eliminate non-functional capsids. Purification usually comprises a purification step for the removal of cells and cellular debris (e.g., using differential centrifugation, density centrifugation and/or filtration) and one or more downstream chromatography steps to separate the AAV product from various impurities in the clarified cell culture feed. However, until the present invention, it was not possible to consistently remove non-functional capsids (i.e., deamidated empty capsids, deamidated partial capsids, or deamidated full capsids) to less than about 10% of the total capsids solely by using ultracentrifugation methods or AEX (without a fatty acid mobile phase) methods. This problem hampered utilization of high-throughput systems for production of commercial quantities of AAV product with sufficient purity and potency. Solving this problem provides an AAV product with more transgene expression capability (i.e., potency) given the higher ratio of functional capsids to non-functional capsids and less immunoreactivity in an effective dose given the reduced number of non-functional capsids. To achieve an AAV product of this purity, the greater negative charge of full capsids and the less negative charge of non-functional capsids is exploited. See FIGS. 1A and 1B. Notably, the negative charge correlates with hydrophobicity. The greater the negative charge, the more hydrophobic. Thus, AEX conditions can be manipulated to remove empty capsids before the main peak of functional capsids; however, a tailing “non-product” peak results unless the elution conditions (e.g., hydrophobicity) are modified to keep the non-product peak attached to the resin. The tailing “non-product” peak consists primarily of deamidated empty capsids and, to a lesser extent, deamidated capsids containing a partial transgene and deamidated full capsids. The non-product peak also contains more than 50% deamidated VP1 than the product peak containing functional transgenes. Deamidation imparts a negative charge on the N terminus of VP1, which is very hydrophobic, resulting in a loss of potency and a drop in transgene expression. See FIGS. 2A and 2B. Baseline resolution between the product peak and non-product peak is not achievable using traditional AEX conditions. Conditions that promote retention of the non-functional capsids results in elution of an AAV product having less than 10% non-functional capsids.

Thus, provided herein is an AAV product comprising functional capsids containing a transgene and less deamidated AAV capsids as compared to a control AAV product. The AAV product comprises functional capsids, wherein the functional capsids contain a transgene expressible in a host cell, and less deamidated capsids than a control AAV product purified solely by ultracentrifugation or by AEX without a fatty acid mobile phase. In some embodiments, there is a decrease of at least 99%, 95% 90%, 85%, 80%, 75%, 70%, 65%, 60%, 55%, or 50% in the number of deamidated AAV capsids as compared to an AAV product control. As used throughout, a control AAV product comprises AAV capsids that have the same serotype and comprise the same transgene as compared to an AAV product described herein, but the control AAV product is purified by a different process (e.g., ultracentrifugation or AEX without a fatty acid mobile phase).

Also provided herein is an AAV product comprising functional capsids, wherein the functional capsids contain a transgene expressible in a host cell and wherein the product is less hydrophobic than a control AAV product purified solely by ultracentrifugation or by AEX without a fatty acid mobile phase.

As used herein, a functional capsid is a full or nearly full AAV capsid comprising a transgene, wherein the full or nearly full AAV capsid expresses a functional gene product encoded by the transgene in a host cell. Functional capsids are also referred to as functional recombinant AAV (rAAV) virions or functional rAAV particles. As used herein, a full capsid comprises the full length of the desired genetic material, i.e., the transgene; a nearly full capsid comprises genetic material sufficient to express a functional protein encoded by the transgene once introduced into the host cell; partially full capsids contain a portion of the transgene but the portion is insufficient for expression of the transgene in a host cell; and empty capsids are devoid or relatively devoid of functional genetic material, i.e., the transgene. As used herein, the term non-functional capsid refers to a deamidated capsid that fails to express a transgene in a host cell. As used herein, full and nearly full capsids amidated at position N57 of VP1, which are capable of expressing a transgene in a host cell, are referred to collectively as “functional” capsids.

As used throughout, an AAV product is a composition comprising a plurality of AAV capsids. In some embodiments, the AAV product comprises at least about 90%, 91%, 92%, 93%, 94%, 95%, 96%, 97%, 98%, 99%, or 100% functional capsids. In some embodiments, the AAV product comprises, at least about 90%, 91%, 92%, 93%, 94%, 95%, 96%, 97%, 98%, 99%, or 100% full or nearly full capsids. In some embodiments, the AAV product comprises, less than about 10%, 9%, 8%, 7%, 6%, 5%, 4%, 3%, 2%, 1% non-functional capsids.

In the AAV products and methods described herein, non-functional capsids are more hydrophobic than functional capsids. This hydrophobicity in non-functional capsids is due at least in part to deamidation. Such deamidation, for example, is at position N57 of VP1. Without meaning to be limited by theory, capsids that are misconfigured may have more N57 of VP1 extruded from or exposed on the surface of non-functional capsids, and such exposure may allow for deamidation. In some embodiments, the AAV products provided herein are relatively devoid of deamidated N57. For example, the AAV product can comprise less than 10% deamidated N57 on VP1 of the capsids, a level lower than that achieved solely with ultracentrifugation or AEX (without a fatty acid mobile phase). An exemplary VP1 capsid sequence is set forth under GenBank Accession No. QEU45513.1. See also FIG. 2A showing sequences of VP1 for various AAV serotypes.

In some embodiments, the AAV product has a lower ratio of VP1 to VP2 than a control AAV product purified solely by ultracentrifugation or AEX (without fatty acid mobile phase). For example, a 1:1 ratio of VP1:VP2 in AAV products made according to methods known in the art would be a smaller ratio (i.e., <1:1 VPL:VP2) when hydrophobic capsids (e.g., those having deamidated VP1) are removed.

The amount of deamidation in any of the products described herein can be detected by methods known in the art, for example, liquid chromatography or mass spectrometry.

Optionally, the AAV product consists essentially of functional capsids containing a transgene and a buffer. As used herein, consists of, or consisting essentially of functional capsids, means the AAV product comprises less than 10% empty capsids.

In some embodiments, the AAV product has greater transgene expression in a host cell than a control AAV product purified solely by ultracentrifugation or AEX without fatty acid mobile phase. Any of the AAV products disclosed herein can be introduced into cells (e.g., using any techniques known in the art) for expression of a protein(s) encoded by the transgene in the AAV product. Accordingly, provided herein is a recombinant cell and populations of recombinant cells comprising an AAV product disclosed herein.

In some AAV products, the transgene can be a DNA or RNA sequence that encodes a therapeutic product. Upon introduction into a host cell, the DNA sequence can be transcribed into a target RNA molecule. Alternatively, the transgene can be a target RNA molecule. Target RNA molecules include, without limitation, miRNA, shRNA, siRNA, antisense RNA, gRNA, antagomirs, miRNA sponges, RNA aptazymes, RNA aptamers, mRNA, lncRNAs, ribozymes, and synthetic RNAs known in the art.

In some embodiments, the transgene encodes one or more polypeptides or a functional fragment thereof. Such transgenes can comprise the complete coding sequence of a polypeptide or only a fragment of a coding sequence of a polypeptide. In certain embodiments, the transgene encodes a polypeptide that is useful to treat a disease or disorder in a subject. Suitable polypeptides include, without limitation, 0-globin; hemoglobin; tissue plasminogen activator; coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-a receptors, soluble interleukin receptors (e.g., soluble IL-1 receptors and soluble type II IL-1 receptors), soluble γ/Δ T cell receptors, ligand-binding fragments of a soluble receptor, and the like; enzymes, such as a-glucosidase, imiglucerase, β-glucocerebrosidase, and alglucerase; enzyme activators, such as tissue plasminogen activator; chemokines, such as IP-10, monokine induced by interferon-gamma (Mig), Groα/IL-8, RANTES, MIP-1α, MIP-1β, MCP-1, PF-4, and the like; angiogenic agents, such as vascular endothelial growth factors (VEGFs, e.g., VEGF121, VEGF165, VEGF-C, VEGF-2), glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastrin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagon, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); tissue factors; macrophage activating factors; tumor necrosis factor (TNF); neutrophil chemotactic factor (NCF); tissue inhibitors of metalloproteinases; vasoactive intestinal peptide; angiogenin; angiotropin; fibrin; hirudin; IL-1 receptor antagonists; ciliary neurotrophic factor (CNTF); brain-derived neurotrophic factor (BDNF); neurotrophins 3 and 4/5 (NT-3 and -4/5); glial cell derived neurotrophic factor (GDNF); aromatic amino acid decarboxylase (AADC); Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase, glucose transporter, aldolase A, β-enolase, glycogen synthase; lysosomal enzymes, such as iduronate-2-sulfatase (12S), and arylsulfatase A; and mitochondrial proteins, such as frataxin.

In some embodiments, the transgene encodes a protein defective in a subject with one or more lysosomal storage diseases. Suitable proteins include, without limitation, α-sialidase, cathepsin A, α-mannosidase, β-mannosidase, glycosylasparaginase, α-fucosidase, α-N-acetylglucosaminidase, μ-galactosidase, μ-hexosaminidase α-subunit, μ-hexosaminidase μ-subunit, GM2 activator protein, glucocerebrosidase, Saposin C, Arylsulfatase A, Saposin B, formyl-glycine generating enzyme, β-galactosylceramidase, α-galactosidase A, iduronate sulfatase, α-iduronidase, heparan N-sulfatase, acetyl-CoA transferase, N-acetyl glucosaminidase, β-glucuronidase, N-acetyl glucosamine 6-sulfatase, N-acetylgalactosamine 4-sulfatase, galactose 6-sulfatase, hyaluronidase, α-glucosidase, acid sphingomyelinase, acid ceramidase, acid lipase, cathepsin K, tripeptidyl peptidase, palmitoyl-protein thioesterase, cystinosin, sialin, UDP-N-acetylglucosamine, phosphotransferase γ-subunit, mucolipin-1, LAMP-2, NPC1, CLN3, CLN 6, CLN 8, LYST, MYOV, RAB27A, melanophilin, and AP3 β-subunit.

In some embodiments, the transgene encodes an antibody or a fragment thereof (e.g., a Fab, scFv, or full-length antibody). Suitable antibodies include, without limitation, muromonab-cd3, efalizumab, tositumomab, daclizumab, nebacumab, catumaxomab, edrecolomab, abciximab, rituximab, basiliximab, palivizumab, infliximab, trastuzumab, adalimumab, ibritumomab tiuxetan, omalizumab, cetuximab, bevacizumab, natalizumab, panitumumab, ranibizumab, eculizumab, certolizumab, ustekinumab, canakinumab, golimumab, ofatumumab, tocilizumab, denosumab, belimumab, ipilimumab, brentuximab vedotin, pertuzumab, raxibacumab, obinutuzumab, alemtuzumab, siltuximab, ramucirumab, vedolizumab, blinatumomab, nivolumab, pembrolizumab, idarucizumab, necitumumab, dinutuximab, secukinumab, mepolizumab, alirocumab, evolocumab, daratumumab, elotuzumab, ixekizumab, reslizumab, olaratumab, bezlotoxumab, atezolizumab, obiltoxaximab, inotuzumab ozogamicin, brodalumab, guselkumab, dupilumab, sarilumab, avelumab, ocrelizumab, emicizumab, benralizumab, gemtuzumab ozogamicin, durvalumab, burosumab, erenumab, galcanezumab, lanadelumab, mogamulizumab, tildrakizumab, cemiplimab, fremanezumab, ravulizumab, emapalumab, ibalizumab, moxetumomab, caplacizumab, romosozumab, risankizumab, polatuzumab, eptinezumab, leronlimab, sacituzumab, brolucizumab, isatuximab, and teprotumumab.

In some embodiments, the transgene encodes a nuclease. Suitable nucleases include, without limitation, zinc fingers nucleases (ZFN) (see, e.g., Porteus and Baltimore (2003) Science 300: 763; Miller et al. (2007) Nat. Biotechnol. 25:778-785; Sander et al. (2011) Nature Methods 8:67-69; and Wood et al. (2011) Science 333:307, each of which is hereby incorporated by reference in its entirety), transcription activator-like effectors nucleases (TALEN) (see, e.g., Wood et al. (2011) Science 333:307; Boch et al. (2009) Science 326:1509-1512; Moscou and Bogdanove (2009) Science 326:1501; Christian et al. (2010) Genetics 186:757-761; Miller et al. (2011) Nat. Biotechnol. 29:143-148; Zhang et al. (2011) Nat. Biotechnol. 29:149-153; and Reyon et al. (2012) Nat. Biotechnol. 30(5): 460-465, each of which is hereby incorporated by reference in its entirety), homing endonucleases, meganucleases (see, e.g., U.S. Patent Publication No. US 2014/0121115, which is hereby incorporated by reference in its entirety), and RNA-guided nucleases (see, e.g., Makarova et al. (2018) The CRISPR Journal 1(5): 325-336; and Adli (2018) Nat. Communications 9:1911, each of which is hereby incorporated by reference in its entirety).

In some embodiments, the transgene encodes an RNA-guided nuclease. Suitable RNA-guided nucleases include, without limitation, Class I and Class II clustered regularly interspaced short palindromic repeats (CRISPR)-associated nucleases. Class I is divided into types I, III, and IV, and includes, without limitation, type I (Cas3), type I-A (Cas8a, Cas5), type I-B (Cas8b), type I-C(Cas8c), type I-D (Cas10d), type I-E (Cse1, Cse2), type I-F (Csy1, Csy2, Csy3), type I-U (GSU0054), type III (Cas10), type III-A (Csm2), type III-B (Cmr5), type III-C (Csx10 or Csx11), type III-D (Csx10), and type IV (Csf1). Class II is divided into types II, V, and VI, and includes, without limitation, type II (Cas9), type II-A (Csn2), type II-B (Cas4), type V (Cpf1, C2c1, C2c3), and type VI (Cas13a, Cas13b, Cas13c). RNA-guided nucleases also include naturally-occurring Class II CRISPR nucleases such as Cas9 (Type II) or Cas12a/Cpf1 (Type V), as well as other nucleases derived or obtained therefrom. Exemplary Cas9 nucleases that may be used in the present invention include, but are not limited to, S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9).

In some embodiments, the transgene encodes one or more reporter sequences, which upon expression produce a detectable signal. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins, including, for example, CD2, CD4, CD8, the influenza hemagglutinin protein, and fusion proteins comprising a membrane bound protein appropriately fused to an antigen tag domain from, among others, hemagglutinin or Myc.

### Methods of Reducing Non-Functional Capsids in the AAV Product

Provided herein are methods of reducing the number of non-functional capsids in an AAV product by limiting the post-transfection time target cells are maintained in the bioreactor before the unseparated capsids are isolated and purified and/or by removing at least a sub-population of non-functional capsids from an adenovirus-associated virus (AAV) product comprising separating functional capsids containing a transgene from a subpopulation of non-functional capsids based on hydrophobicity. For example, the time that the cells are retained in a bioreactor is optionally maintained for about two days post-transfection, and purification is performed using AEX (with or without a fatty acid mobile phase and with or without a mixed-mode resin) or HIC. In certain embodiments, purification is performed using AEX with a fatty acid mobile phase or mixed resin or using HIC without limiting the time the cells are present in the bioreactor.

Also provided herein are methods of reducing the number of non-functional capsids in an AAV product by shifting the post-transfection duration and/or temperature at which the cells are maintained in the bioreactor. The time that the transfected cells are retained in a bioreactor is about 2 days (i.e., about 48 hours after transfection). For example, about 2 days may include about 1.5 days to about 2.5 days, include for example, 1.5 days, 1.6 days, 1.7 days, 1.8 days, 1.9 days, 2 days, 2.1 days, 2.2 days, 2.3 days, 2.4 days, and 2.5 days. Optionally, the post-transfection temperature is set to about 30-38° C. (e.g., about 30° C., about 31° C., about 32° C., about 33° C., about 34° C., about 35° C., about 36° C., about 37° C., or about 38° C.). As used herein, about a defined temperature refers to ±0.5° C., for example, about 37° C. may include 36.5° C., 36.6° C., 36.7° C., 36.8° C., 36.9° C., 37.0° C., 37.1° C., 37.2° C., 37.3° C., 37.4° C., or 37.5° C. By way of example, the time that the transfected cells are retained in a bioreactor is optionally about 2 days and the post transfection temperature is optionally set to about 37° C. The post-transfection temperature shift (e.g., to about 37° C.) is performed before the unseparated capsids are isolated and purified. For example, the post-transfection temperature may be about 30° C. to about 38° C. In some embodiments, the post-transfection temperature at which the cells are maintained in the bioreactor is about 37° C. After the transfected cells have been in the bioreactor for about 2 days, the transfected cells are lysed and purified. Purification can then be performed using AEX (with or without a fatty acid mobile phase and with or without a mixed-mode resin) or HIC. In certain embodiments, purification is performed using AEX with a fatty acid mobile phase or mixed resin or using HIC without shifting the post-transfection temperature of the bioreactor.

Existing AAV purification methods, for example, ultracentrifugation, are associated with loss of AAV product yield, are not scalable, and are not amenable to high-throughput processing, often resulting in AAV products containing unacceptable levels of non-functional capsids. Further, existing methods are often hampered by the inability to efficiently separate functional AAV capsids from AAV product-related impurities (i.e., non-functional capsids). The product-related impurities, however, can contribute to immune responses in a host to capsid proteins. The present methods optimize the concentration of functional capsids by removing the non-functional capsids. Additionally, the methods reduce the immunogenicity of an effective dose of the AAV product by removal of capsid proteins associated with non-functional capsids.

Provided herein is a method of removing at least a sub-population of non-functional capsids from an adenovirus-associated virus (AAV) product comprising separating functional capsids containing a transgene from a subpopulation of non-functional capsids based on hydrophobicity. The purification methods target hydrophobic capsids, e.g., capsids with deamidated VP1, for removal to form a final AAV product relatively devoid of deamidated capsids. In particular, capsids that are deamidated at position N57 of VP1 are non-functional capsids. As used herein, removal of the subpopulation of non-functional capsids includes removal of at least 90%, 91%, 92%, 93%, 94%, 95%, 96%, 97%, 98%, 99% or 100% of non-functional capsids. For example, the removed capsids can result in removal of more than 10%, 20%, 30%, 40%, 50%, 60%, 70%, 80%, or 90% of the capsids with deamidated N57 on VP1.

As described herein, non-functional capsids can be separated from functional capsids (i.e., capsids containing a functional transgene and lacking a deamidated N57 on VP1) based on the hydrophobicity of the non-functional capsids. The hydrophobicity of the non-functional capsids is related at least in part to deamidation of VP1 (for example, at position N57 of VP1), which is amidated in functional capsids.

As used throughout, deamidation is a chemical reaction in which an amide functional group in asparagine or glutamine in a polypeptide (e.g., VP1) is converted to another functional group, for example, aspartic acid or glutamic acid, thus introducing a negative charge into the protein. Deamidation of VP1 also results in increased hydrophobicity of non-functional capsids as compared to functional capsids. By using the difference in deamidation (and hydrophobicity) between functional capsids and non-functional capsids, deamidated capsids can be separated from a population of AAV capsids comprising functional capsids to produce an AAV product having fewer non-functional capsids, for example, less than 10%, 9%. 8%, 7%, 6%, 5%, 4%, 3%, 2%, or 1% non-functional capsids, as compared to an AAV product purified solely by ultracentrifugation or by AEX without a fatty acid mobile phase.

Separation based on hydrophobicity can optionally be coupled with separation based on charge. For example, IC can be performed followed by AEX (with or without a fatty acid mobile phase).

By separating and removing the non-functional capsids based on hydrophobicity or based on hydrophobicity and charge, the resulting AAV product has greater potency at a given concentration of capsids than a comparable AAV product in which non-functional capsids are not removed based on hydrophobicity or hydrophobicity and charge. Increased capsid potency means increased transgene expression upon introduction of a given concentration of the AAV product into a host cell. The increased capsid potency was surprising given the reduced ratio of VP1:VP2, as VP1 is usually associated with more capsid infectivity. However, deamidation of VP1 (e.g., at position N57) correlates with a decreased capsid infectivity, making the deamidated capsids a suitable target for eliminating AAV product related impurities. The purification methods described herein provide increased functional capsid yield as well as increased potency of a product at a given concentration of capsids, as there are more functional AAV capsids as compared to AAV products made using existing methods for AAV purification. Further, by removing deamidated capsids, the ratio of VP1:VP2:VP3 can be reduced from about 1:1:8 to 1:1:10, which are typical ratios for capsid formation, to <1:1:8 to <1:1:10.

The methods provided herein comprise one or more chromatography steps in which a source of AAV product is loaded onto, flows through, or is applied to a chromatography substrate to result in differential separation of various components. The source of AAV product can be a cell culture media containing the AAV product or material comprising the AAV product isolated from pre-chromatography steps. It is understood that material eluted in a chromatography step can be applied to a subsequent chromatography substrate to obtain a further purified AAV product.

Separating functional capsids containing the functional transgene from the subpopulation of non-functional capsids comprises a HIC step, an AEX step with a fatty acid mobile phase, or a mixed-mode AEX step. These techniques separate biomolecules based on their surface hydrophobicity differences. Other methods that separate based on hydrophobicity could be used instead of or in addition to these methods.

The HIC step can include one or more HIC steps. HIC methods are known in the art and an exemplary method is described in the Examples. HIC chromatography media contain hydrophobic ligands such as, for example, linear hydrocarbons (e.g., propyl, butyl, hexyl, or octyl). Salts, for example, ammonium sulfate, are used to enhance hydrophobic interactions and the addition of salts drives the capture of proteins onto HIC media. For this reason, HIC resins are usually loaded under high salt concentrations and eluted at lower salt concentrations. One of skill in the art would appreciate that the concentration of salt (e.g., ammonium sulfate) can be manipulated to achieve optimal concentrations for binding and elution. HIC can be used in conjunction with an AEX step (i.e., an AEX step with or without a fatty acid mobile phase). Optionally, HIC is performed before AEX.

In some embodiments, separating functional capsids containing the functional transgene from the subpopulation of non-functional capsids comprises one or more AEX steps, wherein at least one AEX step is performed with a fatty acid mobile phase. In some embodiments, the fatty acid mobile phase comprises octanoic acid or heptanoic acid. The fatty acid imparts a negative charge to the capsids having an exposed VP1, which enables the non-functional capsids to bind tighter to the chromatography resin and show better separation from the functional capsids. Anion exchange chromatography methods are described in the Examples and are known to those of skill in the art. See, for example, U.S. Pat. Nos. 6,008,036, 6,586,226, 5,837,520, 6,261,823, 6,537,793, and International Patent Application Publication Nos. WO 00/50573, WO 02/44348 and WO 03/078592, the contents of which are incorporated herein in their entireties.

In some embodiments, separating functional capsids containing the functional transgene from the subpopulation of non-functional capsids comprises a mixed-mode chromatography step. Such a step optionally utilizes a mixed mode resin that includes a hydrophobic resin. As used herein, the term mixed mode or multimodal refers to a chromatographic substrate that has the capacity for more than one separation mode. For example, mixed-mode chromatography can comprise separating molecules based on hydrophobicity and charge. Mixed mode chromatography methods are set forth in the Examples. Other methods are known to those of skill in the art. See for example, Lesellier et al., “Mixed-Mode Chromatography-A Review,” LCGC Supplements 30(6): 22-33 (2107); and Zhang and Liu, “Mixed-mode chromatography in pharmaceutical and biopharmaceutical applications,” Journal of Pharmaceuticals and Biomedical Analysis 28(5): 73-88 (2016). Both references are incorporated herein in their entireties. Mixed mode resins are commercially available and include, for example, Nuvia aPrime 4A. It is within the skill in the art to determine the resin and format for mixed-mode use.

The methods provided herein enables better separation of functional and non-functional AAV capsids. By eliminating the non-functional capsids, the heterogeneity of the final AAV product is reduced. These methods provide an AAV product having less than 10% non-functional capsids and a more potent product. By removing the non-functional capsids, the effective dose of the AAV product has reduced immunogenicity compared to an effective dose of a product that includes non-functional capsids comprising immunogenic capsid proteins.

### Methods of Treatment

Also provided herein is a method of treating a subject in need of an RNA or polypeptide provided by or produced by a transgene by administering to the subject an effective amount of any of the AAV products described herein, including any AAV product produced by any of the methods of producing an AAV product as described herein.

The transgene contained in the functional capsids of the AAV product can be heterologous to a cell in the subject. As used herein, the phrase heterologous refers to what is not normally found in nature. As such, a heterologous nucleotide sequence may be (a) foreign to its host cell (i.e., is exogenous to the cell); (b) naturally found in the host cell (i.e., endogenous) but present at an unnatural quantity in the cell (i.e., greater or lesser quantity than naturally found in the host cell); or (c) be naturally found in the host cell but positioned outside of its natural locus.

The term effective amount, as used throughout, is defined as any amount necessary to produce a desired physiologic response, for example, reducing or delaying one or more effects or symptoms of a disease or disorder. Effective amounts and schedules for administering any of the AAV products described herein can be determined empirically and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, unwanted cell death, and the like. Generally, the dosage will vary with the species, age, body weight, general health, sex and diet of the subject, the mode and time of administration and severity of the particular condition and can be determined by one of skill in the art. The dosage can be adjusted by the individual physician in the event of any contraindications. Dosages can vary and can be administered in one or more doses.

An effective amount of any of the AAV products described herein will vary and can be determined by one of skill in the art through experimentation and/or clinical trials. For example, an effective dose can be from about 10⁶ to about 10¹⁸ recombinant rAAV virions, or any values in between this range, for example, about 10⁶, 10⁷, 10⁸, 10⁹, 10¹⁰, 10¹¹, 10¹², 10¹³, 10¹⁴, 10¹¹, 10¹⁶, 10¹⁷, 10¹⁸ recombinant AAV particles. Thus, the number of rAAV particles administered to a subject may be on the order ranging from about 10⁶ to 10¹⁸ vector genomes (vgs)/ml, such as for example, about 10⁶, 10⁷, 10⁸, 10⁹, 10¹⁰, 10¹¹, 10¹², 10¹³, 10¹⁴, 10¹⁵, 10¹⁶, 10¹⁷, 10¹⁸ vg/ml. In some embodiments, the number of rAAV particles administered to a subject can be from about 10⁶ to 10¹⁵ vg/kg, or any values in between these amounts, such as for example, about 10⁶, 10⁷, 10⁸, 10⁹, 10¹⁰, 10¹¹, 10¹², 10¹³, 10¹⁴, or 10¹¹ vg/kg. Other effective dosages can be readily established by one of ordinary skill in the art through routine trials establishing dose response curves. Using the AAV products described herein, a lower dose of the AAV product is required as compared to an AAV product made using traditional purification methods.

The compositions described herein are administered in a number of ways depending on whether local or systemic treatment is desired. The compositions are administered via any of several routes of administration, including intraparenchymal injection, intravenously, intrathecally, intramuscularly, intracisternally, intracoronary injection, intramyocardially injection, intradermally, endomyocardiac injection, or a combination thereof. Effective doses for any of the administration methods described herein can be extrapolated from dose-response curves derived from in vitro or animal model test systems.

As used throughout, subject refers to an individual. The subject can be an adult subject or a pediatric subject. Pediatric subjects include subjects ranging in age from birth to eighteen years of age. Preferably, the subject is an animal, for example, a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

As used throughout, treat, treating, and treatment refer to a method of reducing or delaying one or more effects or symptoms of a disease or disorder. The subject can be a patient diagnosed with the disease or disorder. Treatment can also refer to a method of reducing the underlying pathology rather than just the symptoms. The effect of the administration to the subject can have the effect of, but is not limited to, reducing one or more symptoms of the disease, a reduction in the severity of the disease, the complete ablation of the disease, or a delay in the onset or in the worsening of one or more symptoms. For example, a disclosed method is considered to be a treatment if there is about a 10% reduction in one or more symptoms of the disease when compared to the subject prior to treatment or when compared to a control subject or control value. Thus, the reduction can be about a 10, 20, 30, 40, 50, 60, 70, 80, 90, 100%, or any amount of reduction in between.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### Additional Definitions

Unless defined otherwise, all technical and scientific terms used herein have the same meaning as commonly understood by one of ordinary skill in the art to which this disclosure belongs.

Articles “a” and “an”, are used herein to refer to one or to more than one (i.e. at least one) of the grammatical object of the article. By way of example, a nucleic acid sequence means at least one nucleic acid sequence and can include more than one nucleic acid sequence.

The use of any and all examples or exemplary language (e.g., such as) provided herein, is intended merely to better illustrate the invention and does not pose a limitation on the scope of the invention unless otherwise claimed.

The terms may, can, and related terms are intended to convey that the subject matter involved is optional (that is, the subject matter is present in some examples and is not present in other examples), not a reference to a capability of the subject matter or to a probability, unless the context clearly indicates otherwise.

The term about is used to provide flexibility to a numerical range endpoint by providing that a given value may be slightly above or slightly below the endpoint without affecting the desired result.

The terms optional and optionally mean that the subsequently described event, circumstance, or material may or may not occur or be present, and that the description includes instances where the event, circumstance, or material occurs or is present as well as instances where it does not occur or is not present.

The use herein of the terms including, comprising, or having, and variations thereof, is meant to encompass the elements listed thereafter and equivalents thereof as well as additional elements. Embodiments recited as including, comprising, or having certain elements are also contemplated as consisting essentially of and consisting of those certain elements. As used herein, and/or refers to and encompasses any and all possible combinations of one or more of the associated listed items, as well as the lack of combinations were interpreted in the alternative (“or”).

### Embodiments

1. An adenovirus-associated virus (AAV) product comprising
(a) functional capsids, wherein the functional capsids contain a transgene; and
(b) less deamidated capsids than a control AAV product purified solely by ultracentrifugation or by anion-exchange chromatography (AEX) without a fatty acid mobile phase.

2. The AAV product of embodiment 1, wherein the AAV product comprises less N57 deamidated capsids than the control AAV product purified solely by AEX without a fatty acid mobile phase.

3. The AAV product of embodiment 1 or 2, wherein the AAV product comprises less than 10% deamidated N57 on VP1 of the capsids.

4. The AAV product of embodiment 3, wherein the AAV product comprises less than 5% deamidated N57 on VP1 of the capsids.

5. The AAV product of embodiment 4, wherein the AAV product comprises less than 1% deamidated N57 on VP1 of the capsids.

6. The AAV product of any one of embodiments 1-5 wherein the AAV product comprises less than 10% non-functional capsids.

7. The AAV product of any one of embodiments 1-6, wherein the AAV product has greater transgene expression in a host cell than a control AAV product purified solely by ultracentrifugation or AEX without fatty acid mobile phase.

8. The AAV product of any one of embodiments 1-7, wherein the AAV product has a lower ratio of VP1 to VP2 than a control AAV product purified solely by ultracentrifugation or AEX without fatty acid mobile phase.

9. The AAV product of any one of embodiments 1-8, wherein deamidation amount is detected by liquid chromatography-mass spectrometry.

10. An adenovirus-associated virus (AAV) product consisting essentially of functional capsids containing a transgene.

11. The AAV product of embodiment 10, wherein the functional capsids are separated from a population of AAV capsids comprising functional capsids and deamidated capsids to produce the AAV product.

12. The AAV product of embodiment 10 or 11, wherein the AAV product is relatively devoid of capsids with VP1 having a deamidated N57.

13. An adenovirus-associated virus (AAV) product comprising functional capsids, wherein the functional capsids contain a transgene and wherein the product is less hydrophobic than a control AAV product purified solely by ultracentrifugation or by anion-exchange chromatography (AEX) without a fatty acid mobile phase.

14. The AAV product of embodiment 13, wherein the AAV product comprises less than 10% non-functional capsids.

15. The AAV product of embodiment 13 or 14, wherein the AAV product has greater transgene expression in a host cell than a control AAV product purified solely by ultracentrifugation or AEX without fatty acid mobile phase.

16. The AAV product of any one of embodiments [0007]-15, wherein the AAV product has a lower ratio of VP1 to VP2 than a control AAV product purified solely by ultracentrifugation or AEX without fatty acid mobile phase.

17. An adenovirus-associated virus (AAV) product comprising capsids, wherein the AAV product comprises less than 10% deamidated N57 on VP1 of the capsids.

18. The AAV product of embodiment 17, wherein the AAV product comprises less than 5% deamidated N57 on VP1 of the capsids.

19. The AAV product of embodiment 18, wherein the AAV product comprises less than 1% deamidated N57 on VP1 of the capsids.

20. An adenovirus-associated virus (AAV) product comprising AAV capsids containing a transgene, wherein the AAV product has greater transgene expression in a host cell than a control AAV product purified solely by ultracentrifugation or AEX without fatty acid mobile phase.

21. An adenovirus-associated virus (AAV) product comprising AAV capsids containing a transgene, wherein the AAV product has a lower ratio of VP1 to VP2 than a control AAV product purified solely by ultracentrifugation or AEX without fatty acid mobile phase.

22. A method of removing at least a sub-population of non-functional capsids from an adenovirus-associated virus (AAV) product comprising separating functional capsids containing a transgene from a subpopulation of non-functional capsids based on hydrophobicity.

23. The method of embodiment 22, wherein separating the functional capsids from the non-functional capsids is further based on charge.

24. The method of embodiment 22 or 23, wherein the removed non-functional capsids comprise deamidated capsids.

25. The method of embodiment 24, wherein the removed deamidated capsids comprise VP1 with deamidated N57.

26. The method of embodiment any one of embodiments 22-25, wherein separating functional capsids containing the functional transgene from the subpopulation of non-functional capsids comprises:
(a) a hydrophobic interaction chromatography (HIC) step,
(b) anion-exchange chromatography step with a fatty acid mobile phase, or
(c) mixed-mode anion-exchange chromatography step.

27. The method of embodiment 26, wherein the separation comprises an HIC step.

28. The method of embodiment 27, wherein the separation further comprises an anion-exchange chromatography (AEX) step.

29. The method of embodiment 28, wherein the AEX step is performed without a fatty acid mobile phase.

30. The method of embodiment 27-29, wherein the HIC step precedes the AEX step.

31. The method of embodiment 26, wherein the separation comprises the anion-exchange chromatography step with a fatty acid mobile phase.

32. The method of embodiment 31, wherein the fatty acid mobile phase comprises octanoic acid or heptanoic acid.

33. The method of embodiment 26, wherein the separation comprises a mixed-mode resin chromatography step.

34. The method of embodiment 33, wherein the mixed mode resin is a hydrophobic anion exchange resin.

35. An AAV product produced by the method of any one of embodiments 22-34.

36. An adenovirus-associated virus (AAV) product comprising:
(a) functional capsids, wherein the functional capsids contain a transgene; and
(b) less hydrophobic capsids than a control AAV product purified solely by ultracentrifugation or by anion-exchange chromatography (AEX) without a fatty acid mobile phase.

37. The AAV product of embodiment 36, wherein the AAV product comprises less N57 deamidated capsids than the control AAV product purified solely by AEX without a fatty acid mobile phase.

38. The AAV product of embodiment 36 or 37, wherein the AAV product comprises less than 10% deamidated N57 on VP1 of the capsids.

39. The AAV product of embodiment 38, wherein the AAV product comprises less than 5% deamidated N57 on VP1 of the capsids.

40. The AAV product of embodiment 39 wherein the AAV product comprises less than 1% deamidated N57 on VP1 of the capsids.

41. The AAV product of any one of embodiments 36-40, wherein the AAV product comprises less than 10% non-functional capsids.

42. The AAV product of any one of embodiments 36-41, wherein the AAV product has greater transgene expression in a host cell than a control AAV product purified solely by ultracentrifugation or AEX without fatty acid mobile phase.

43. The AAV product of any one of embodiments 36-42, wherein the AAV product has a lower ratio of VP1 to VP2 than a control AAV product purified solely by ultracentrifugation or AEX without fatty acid mobile phase.

44. The AAV product of any one of embodiments 36-43, wherein deamidation amount is detected by liquid chromatography-mass spectrometry.

45. An adenovirus-associated virus (AAV) product consisting essentially of functional capsids containing a transgene, wherein the functional capsids are separated from a population of hydrophobic AAV capsids to produce the AAV product.

46. The AAV product of embodiment 45, wherein the AAV product is relatively devoid of capsids with VP1 having a deamidated N57.

47. The AAV product of embodiment 45 or 46, wherein the AAV product comprises less than 10% non-functional capsids.

48. The AAV product of embodiment 45 or 46, wherein the AAV product has greater transgene expression in a host cell than a control AAV product purified solely by ultracentrifugation or AEX without fatty acid mobile phase.

49. The AAV product of any one of embodiments 45-48, wherein the AAV product has a lower ratio of VP1 to VP2 than a control AAV product purified solely by ultracentrifugation or AEX without fatty acid mobile phase.

50. A method of treating a subject in need of a protein produced by a transgene comprising administering to the subject the AAV product of any one of embodiments 1-21 and 35-49.

51. An adenovirus-associated virus (AAV) product relatively devoid of non-functional capsids.

52. An adenovirus-associated virus (AAV) product consisting essentially of functional capsids containing a transgene and a buffer.

53. The method of any one of embodiments 22-34, wherein the AAV capsids are harvested at about 2 days post-transfection.

54. The method of embodiment 53, wherein the post-transfection temperature is about 30° C. to about 38° C.

55. The method of embodiment 54, wherein the post transfection temperature is about 37° C. and wherein the AAV product is harvested at about 2 days post transfection.

56. The method of embodiment 22, wherein separating the functional capsids from the non-functional capsids comprises harvesting AAV capsids at about 2 days post-transfection (e.g., 1.5-2.5 days).

57. The method of embodiment 56, wherein the post-transfection temperature is about 36° C., 37° C., or 38° C.

58. The method of embodiment 57, wherein the post transfection temperature is about 37° C. and wherein the AAV product is harvested at about 2 days post transfection.

### EXAMPLES

### Example 1: Comparing Chromatographic Resolution Between Nuvia aPrime 4A Anion Exchange Column and Poros HQ50 Anion Exchange Column for the Purification of DNA Containing Capsids

### Capsid Preparation

AAV capsids were produced in a 50 L bioreactor using a transient transfection comprised of three plasmids. Cells were lysed after 72 hours to expose the AAV capsids produced during the upstream process. The lysed material was then clarified using depth filtration at constant flux. The product of this process is known as the clarified lysate. The clarified lysate was then captured on an affinity chromatography resin under bind and elute conditions. Briefly, the clarified lysate was loaded onto the resin, washed to remove host cell related impurities, and then eluted under acidic conditions. The affinity eluate was neutralized to pH 8.0 using a tris buffer. This is known as the affinity product. The affinity product was aliquoted and frozen at −80 C. Appropriate volumes of aliquots were thawed at room temperature to be used for the experiments performed and described in this application.

### Sample Prep

AAV capsids (2×10¹⁴ caps/mL), were diluted with Load Dilution Buffer (28 mM Ammonium Acetate, 2 mM MgCl₂, 0.01% (w/v %) Poloxamer 188) so the final loading concentration was less than 2×10¹³ caps/mL. Sample pH was adjusted to pH 9.3 using either 1N NaOH or 1N acetic acid and conductivity was adjusted to 3 mS/cm using a 0.01% (w/v %) Poloxamer 188, 2 mM MgCl₂ solution.

### Resin Equilibration

A 1 mL Nuvia aPrime 4A anion exchange column (Bio-rad, Hercules, CA, Cat #12007392) and a 1 mL Poros HQ50 anion exchange column (ThermoFisher, Waltham, MA, Cat #PN4481315) were prepared by first washing out the storage ethanol using 10 column volumes (CV) of wash solution (28 mM Ammonium Acetate, 2 mM Magnesium Chloride, 0.01% (w/v %) Poloxamer 188, pH 9.3, Conductivity: 3 mS/cm), followed by 5 CV sanitization (0.5N Sodium Hydroxide) held for 30 minutes. Equilibration was achieved by first pre-equilibrating the column in 1M Ammonium Acetate, pH ˜6.7 to remove chloride ions, and then pumping through 5 CV of equilibration Buffer A (28 mM Ammonium Acetate, 2 mM Magnesium Chloride, 0.01% (w/v %) Poloxamer 188, pH 9.3). All equilibration steps were pumped through the system at a rate of 1 mL/min for 1 minute.

### Anion Exchange (AEX) Chromatography

Diluted sample was applied to both equilibrated columns at 0.33 mL/min for a 3-minutes residence time. Following sample loading, the column was washed with 5 CV Buffer A and then 5 CV of wash buffer (10 mM Ammonium Acetate 0.01% (w/v %) Poloxamer 188, pH 9.3). Column elution was performed using a 60 CV linear gradient from 0-100%, increasing step wise by 0.01%, with Buffer B (1M Ammonium Acetate, pH 9.3) followed by 20 CV at 100% Buffer B with 1 mL fractions being collected into pre-neutralizing buffer. After sample collection, the columns were stripped with 5 CV of stripping buffer (50 mM Tris, 2M Sodium Chloride, 0.01% (w/v %) Poloxamer 188, pH 8), followed by 5 CV of cleaning buffer (0.5N NaOH), then they were pH re-equilibrated using 1M Ammonium Acetate (pH ˜6.7), and finally stored in 5 CV worth of 100 mM Sodium Phosphate, 18% Ethanol (v/v %), pH 7.

### Chromatography Results

A 1 mL prepacked Nuvia aPrime 4A column and Poros HQ50 (Control) column was run for comparison purposes. The operational results can be found in Table 1.

TABLE 1
Chromatography Column Operational Results

| | Nuvia | Poros |
|---|---|---|
| | APrime | HQ50 |
| Output | 4A Result | Results |
| Load ratio (capsids/mL of | 2.00 × 10¹⁴ | 2.00 × 10¹⁴ |
| Resin) | | |
| Pre-dilution volume (mL) | 0.962 mL | 0.962 mL |
| Post-dilution volume (mL) | 42.48 | 43.13 |
| Dilution Factor (x) | 44.15 | 44.83 |
| Estimated Capsids | 4.53 × 10¹² | 4.46 × 10¹² |
| Concentration Post Dilution | | |
| (capsids/ml) | | |
| pH post-dilution | 9.25 | 9.25 |
| Conductivity post-dilution | 3.04 | 2.95 |
| (mS/cm) | | |

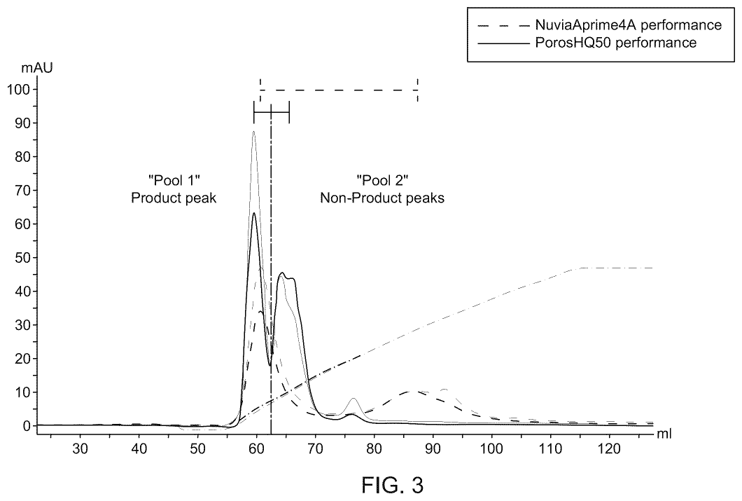

Chromatograms were generated for the Nuvia aPrime 4A run and the control (POROS HQ 50) run. The analysis of these chromatograms showed that Nuvia aPrime 4A was able to provide ample resolution between Pool 1 (mostly full capsid) and Pool 2 (mostly empty capsids). To have a better understanding of chromatographic performance of this new technology, the two chromatograms were overlayed and shown in FIG. 3.

### Data Analysis

The area under the curve of 260 nm and 280 nm absorbance and the ratio of A260/A280 ratios, which is indicative of how many intact and empty capsids are present, was calculated for both the Nuvia aPrime 4A and the Poros HQ50 column. This data is provided in Table 2. Analysis of Table 2 showed that Nuvia aPrime 4A and Poros HQ50 have similar area ratio values and performance for Pool 1 (mostly full capsids). The A260/280 ratio for Nuvia aPrime 4A Pool 2 (mostly empty capsids) and strip was found to be higher compared to Poros HQ50 Pool 2 and strip pools.

TABLE 2
Area under the curve of 260 nm and 280 nm comparison

| | Area | Area | |
|---|---|---|---|
| Pool Type | A260 | A280 | A260/A280 |
| Nuvia aPrime 4A | 307.7 | 226.0 | 1.364 |
| Pool1 | | | |
| PorosHQ50 Q Pool1 | 265.6 | 193.9 | 1.369 |
| Nuvia aPrime 4A | 201.7 | 173.3 | 1.163 |
| Pool2 | | | |
| PorosHQ50 Pool2 | 207.0 | 236.7 | 0.870 |
| Nuvia aPrime 4A | 18.65 | 9.87 | 1.88 |
| Strip PEAK | | | |
| PorosHQ50 Strip | 3.402 | 2.080 | 1.635 |
| PEAK | | | |

### Vector Genome & Capsid Mass Balance Results

The vector genome (VG) mass balance for the two columns is shown in Table 3 and the capsid mass balance is shown in Table 4.

TABLE 3
VG Yield for Nuvia aPrime 4A and Poros HQ50 column

| | Nuvia aPrime 4A VG | Poros HQ50 VG |
|---|---|---|
| Pool Type | Yield (%) | Yield (%) |
| FTW | 12.6% | 0.02% |
| Strip | 0.10% | 0.06% |
| Pool 1 | 56.7% | 62.0% |
| Pool 2 | 8.66% | 16.2% |
| VG Mass Balance | 78.0% | 78.3% |

TABLE 4
Capsid Yield for Nuvia aPrime 4A and Poros HQ50 column

| | Nuvia aPrime 4A Capsid | PorosHQ50 Capsid |
|---|---|---|
| Pool Type | Yield (%) | Yield (%) |
| FTW | 57.99% | 1.72 |
| Strip | 0.12% | 0.05 |
| Pool 1 | 13.37% | 14.4 |
| Pool 2 | 12.13% | 27.1 |
| Capsid Mass Balance | 83.60 | 43.23 |

Analyses of Table 3 and Table 4 showed comparable Pool 1 (Product peak) VG and capsid recoveries for Nuvia aPrime 4A and Poros HQ5 columns. The Pool 2 (mostly empty capsid peak) was found to have lower VG and capsid yield for Nuvia aPrime 4A when compared to Poros HQ50 column. The Flowthrough/wash pool for Nuvia aPrime 4A was found to have higher VG and capsid recovery when compared to Poros HQ50. The VG recovery and capsid recovery for Strip pools for both resins were found to be comparable.

Nuvia aPrime 4A and Poros HQ50 Pool 1 (product peak) and Pool 2 (mostly empty capsid peak) were submitted for Analytical Ultracentrifugation (AUC) profiling. The AUC sample data for Nuvia aPrime 4A Pool 1 and Pool 2 are shown in Table 5 and Table 6, respectively.

TABLE 5
AUC Sample data for Pool 1 of Nuvia a Prime 4A

| Optima-746 | % |
|---|---|
| Empty Capsids | 20.4% |
| Partial Peak 1 | 3.9% |
| Partial Peak 2 | 5.6% |
| Partial Peak 3 | 6.9% |
| All Partial Capsids | 16.4% |
| Full Capsids | 63.1% |

TABLE 6
AUC Sample data for Pool 2 of Nuvia a Prime 4A

| Optima-846 | % |
|---|---|
| Empty Capsids | 81.9% |
| Partial Peak 1 | 4.6% |
| Partial Peak 2 | 3.5% |
| Partial Peak 3 | 5.8% |
| All Partial Capsids | 13.9% |
| Full Capsids | 5.8% |

The AUC profile and sample data for PorosHQ50 Pool 1 and Pool 2 are shown in Table 7 and Table 8, respectively.

TABLE 7
AUC Sample data for Pool 1 of PorosHQ50

| Optima-782 | % |
|---|---|
| Empty Capsids | 8.7% |
| Partial Peak 1 | 2.6% |
| Partial Peak 2 | 7.9% |
| Partial Peak 3 | 7.5% |
| All Partial Capsids | 18.0% |
| Full Capsids | 73.3% |

TABLE 8
AUC Sample data for Pool 2 of Poros HQ50

| Optima-782 | % |
|---|---|
| Empty Capsids | 79.6% |
| Partial Peak 1 | 4.2% |
| Partial Peak 2 | 3.0% |
| Partial Peak 3 | 2.0% |
| Partial Peak 4 | 3.3% |
| All Partial Capsids | 12.4% |
| Full Capsids | 8.0% |

Analysis of AUC profiles for Pool 1 of Nuvia aPrime 4A and Poro sHQ50 showed that Poros HQ50 had a higher percentage of full capsids compared to Nuvia aPrime 4A. The AUC profile for Pool 2 of Nuvia aPrime 4A and PorosHQ50 were also compared. The Pool 2 profiles for both columns were found to be comparable to each other and were mostly composed of empty capsids.

The reduction in recovered full capsids using Nuvia aPrime 4A and Poro sHQ50 is slightly reduced. Without meaning to be limited by theory, this is likely because even some of the full capsids are deamidated and thus non-functional. Removal of the deamidated full capsids is measured as a reduction in full capsids but reflects elimination of full capsids that are non-functional.

### Example 2: Introduction of Fatty Acid into AEX Mobile Phase to Increase Chromatographic Resolution

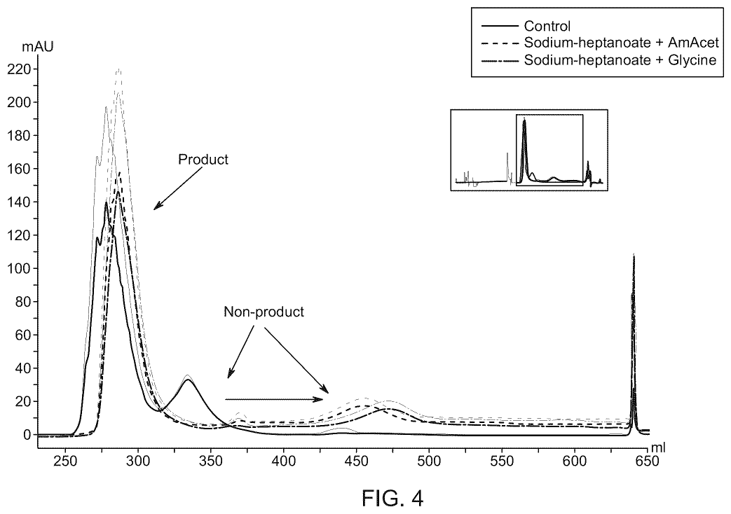

AEX chromatography using POROS HQ50 columns was performed as described above, except the fatty acid heptanoate was incorporated into the mobile phase. As shown in FIG. 4, those chromatography conditions that used heptanoate in the mobile phase showed increased chromatographic resolution between the Pool 1 product peak and the Pool 2 non-product peaks.

### Example 3: Two-Step Chromatography Using Hydrophobic Interaction Chromatography (HIC) Coupled with AEX Chromatography

### Sample Prep

AAV capsids (2.6×10¹⁴ caps/mL), were diluted 7× in Load Dilution Buffer (1.5M Ammonium Sulphate, 100 mM Sodium Phosphate, 0.01% (w/v %) Poloxamer-188, pH 7.0, 187 mS/cm). Salt concentration was adjusted by adding 60% (v/v %) of Salt Spike Buffer (4M Ammonium Sulphate, 100 mM Sodium Phosphate, 0.01% (w/v %) Poloxamer-188) per volume of capsid sample to reach a final salt concentration of 1.5M Ammonium Sulphate. pH and conductivity were measured after sample dilution.

The steps for HIC chromatography and subsequent steps are outlined below.

| | |
|---|---|
| Nuvia aPrime 4A: | Column: 44.2 mL Nuvia aPrime 4A (2.5 cmD × 9 cmL) |
| HIC Chromatography - | Estimated Load Challenge: 2.6 × 10¹⁴ Capsids/mL Resin |
| Gradient Elution | Operating Flowrate: |
| | 22 mL/min (2 min residence time) for steps with no product contact |
| | 11 mL/min (4 min residence time) for Load, Re-equilibration |
| | (wash) and Gradient Elution |
| | HIC Steps: |
| | Ethanol Washout (5 CV): Deionized water |
| | Sanitization (5 CV, Hold for 30 mins): 0.5N Sodium Hydroxide |
| | Washout (5 CV): Deionized water |
| | Equilibration (10 CV): 1.5M Ammonium Sulphate, 100 mM |
| | Sodium Phosphate, 0.01% (w/v %) Poloxamer-188, pH 7.0, |
| | 187 mS/cm |
| | HIC Load |
| | Re-equilibration (5 CV): 1.5M Ammonium Sulphate, 100 mM |
| | Sodium Phosphate, 0.01% (w/v %) Poloxamer-188, pH 7.0, |
| | 187 mS/cm |
| | Linear Gradient Elution (40 CV) - 0-100% B: 100 mM Sodium |
| | Phosphate, 0.01% (w/v %) Poloxamer-188, pH 7.0 |
| | Elution (10 CV) - 100% B: 100 mM Sodium Phosphate, 0.01% |
| | (w/v %) Poloxamer-188, pH 7.0 |
| | Strip (5 CV): Deionized water |
| | Low pH Strip (7 CV): 100 mM Glycine, 100 mM NaCl, 0.01% |
| | (w/v %) Poloxamer-188, pH 2.5 |
| | Sanitization (7 CV): 0.5N Sodium Hydroxide with 30 min |
| | incubation |
| | Washout (3 CV): Deionized water |
| | Storage (5 CV): 100 mM Sodium Phosphate, 18% (v/v %) Ethanol, |
| | pH 7.0 |
| | Neutralization: |
| | Neutralize low pH strip with 10% (v/v %) of 1M Tris, pH 9.0, 0.01% |
| | (w/v %) Poloxamer-188 |
| TFF-2 Diafiltration/ | Membrane: MicroKros 40 cm² 100K mPES |
| Concentration of | Estimated Load Challenge: 1.8 × 10¹⁸ capsids/m² (HIC Pool 1) and |
| each HIC Elution | 5.5 × 10¹⁷ capsids/m² (HIC Pool 2) (range 5 × 10¹⁷−2 × 10¹⁸) |
| Pool | Equilibration/Diafiltration Buffer: Neutralized Affinity Buffer |
| | (100 mM Glycine, 100 mM NaCl, pH 3.3, with 2% (v/v %) of 1M |
| | Tris, 100 mM NaCl, pH 10.9) |
| | TFF-2 Load: HIC Gradient Elution Pool 1; and HIC Gradient |
| | Elution Pool 2 |
| | TFF Parameters: Crossflow: 17 mL/min; TMP: 5-9 psi |
| | Diafiltration Volumes: 7-8 DVs |
| | Final Volume Concentration Target: Pool 1: Down to 40 mL |
| | (~1 + 14 capsids/mL); Pool 2: Down to 30 mL (~7 × 10¹³ |
| | capsids/mL) |
| Load Preparation of | Load Dilution Buffer: 28 mM Ammonium Acetate, 2 mm |
| each HIC Elution | Magnesium Chloride, 0.01% (w/v %) Poloxamer-188 pH 9.3 |
| Pools for AEX Run | Conductivity Adjustment Buffer: 0.01% (w/v %) Poloxamer-188, |
| | 2 mM Magnesium Chloride |
| | Load dilution: Dilute sample 39x in load dilution buffer |
| | Cond. Adjustment: Add 12% (v/v %) of Conductivity Adjustment |
| | Buffer to total load volume. |
| | Check that final conductivity is 3.0 mS/cm. Adjust if necessary. |
| | Check pH after dilution is complete. |
| POROS HQ 50: AEX | Column: Prepacked 10 mL POROS HQ 50 (1.3 cmD × 10 cmL) |
| Chromatography of | Estimated Load Challenge: HIC Pool 1: 1.7 × 10¹⁴ Capsids/mL |
| each Concentrated | Resin; HIC Pool 2: 1.1 × 10¹⁴ Capsids/mL Resin |
| HIC Elution Pools - | Operating Flowrate: |
| Gradient Elution | 10 mL/min (1 min residence time) for Ethanol washout, |
| | Conditioning, Sanitization, Equilibration, Load, Wash1, Wash2, |
| | Strip, Cleaning and Storage block |
| | 1.67 mL/min (6 min residence time) for gradient elution |
| | AEX Steps: |
| | Ethanol Washout (10 CV): 10 mM Ammonium Acetate, 0.01% |
| | (w/v %) Poloxamer-188, pH 9.3 |
| | Sanitization (5 CV, Hold for 30 mins): 0.5N Sodium Hydroxide |
| | Conditioning (to remove Cl-ion) (5 CV): 1M Ammonium Acetate, |
| | pH as is (~6.7) |
| | Equilibration (5 CV): 28 mM Ammonium Acetate, 2 mM |
| | Magnesium Chloride, 0.01% (w/v %) Poloxamer-188, pH 9.3, |
| | 3 mS/cm |
| | AEX Load |
| | Re-equilibration (5 CV): 28 mM Ammonium Acetate, 2 mm |
| | Magnesium Chloride, 0.01% (w/v %) Poloxamer-188, pH 9.3, |
| | 3 mS/cm |
| | Wash 2 (5 CV): 10 mM Ammonium Acetate, 0.01% (w/v %) |
| | Poloxamer-188, pH 9.3, 1 mS/cm |
| | Linear Gradient Elution (60 CV) - 0-100% B: 400 mM |
| | Ammonium acetate, 0.01% (w/v %) Poloxamer-188, pH 9.3, |
| | conductivity 24 mS/cm |
| | Elution (20 CV) - 100% B: 400 mM Ammonium acetate, 0.01% |
| | (w/v %) Poloxamer-188, pH 9.3, conductivity 24 mS/cm |
| | Strip (5 CV): 1M Tris, 2M NaCl, 0.01% (w/v %) Poloxamer-188, |
| | pH 8.0 |
| | Sanitization (5 CV, Hold for 30 mins): 0.5N Sodium Hydroxide |
| | Conditioning (to remove Cl-ion) (5 CV): 1M Ammonium Acetate, |
| | pH as is (~6.7) |
| | Wash 2 (5 CV): 10 mM Ammonium Acetate, 0.01% (w/v %) |
| | Poloxamer-188, pH 9.3, 1 mS/cm |
| | Storage (5 CV): 100 mM Sodium Phosphate, 18% (v/v %) Ethanol |
| | pH 7.0 |
| | Neutralization: |
| | Neutralize FTW and Elution Pools with 11% v/v Strip Buffer (1M |
| | Tris, 2M NaCl, 0.01% (w/v %) Poloxamer-188) |

### HIC Chromatography

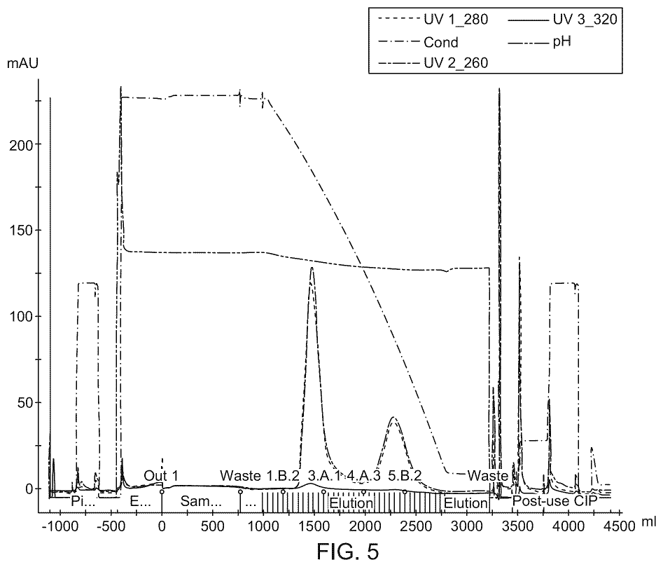

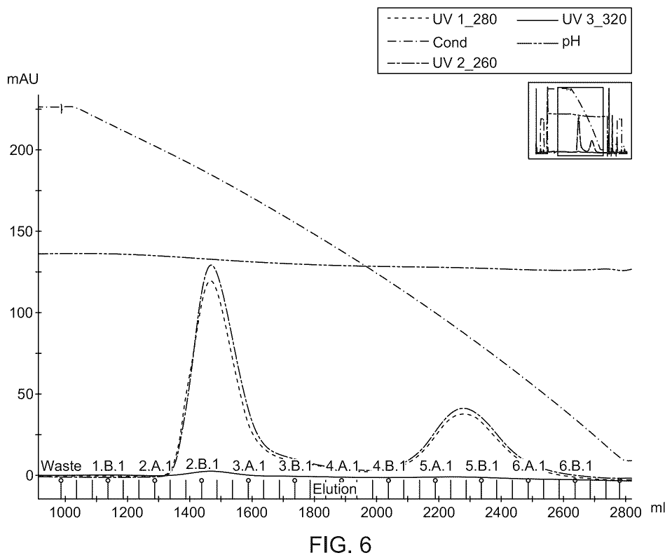

A previously packed 44 mL Nuvia aPrime 4A column by Bio-Rad was used and challenged to 2.6×10¹⁴ capsids/mL of resin. The load was prepared by diluting the AF Product 7× in a 1.5M Ammonium Sulphate Load Dilution Buffer, then adding 60% (v/v %) of a 4M Ammonium Sulphate Salt Spike Buffer per volume of AF Product used. The chromatogram of this HIC mode run, and the respective gradient elution are shown in FIGS. 5 and 6, respectively.

The final conductivity of the Load material was 189 mS/cm, close to the conductivity of the 1.5M Ammonium Sulphate Equilibration Buffer (188 mS/cm). With this load preparation method for the HIC run, no breakthrough was observed up to 2.6×10¹⁴ capsids/mL of resin (FIG. 5; see also, FIG. 6).

Gradient elution with decreasing conductivity separated the material in two distinct peaks, which were collected into two pools. Material in HIC Pool 1 eluted first, at higher conductivity, and was less hydrophobic. HIC Pool 1 elution, measured by the ATKA, started at 188 mS/cm and peaked at 172 mS/cm. Material in HIC Pool 2 was more tightly bound to the resin, being more hydrophobic than the HIC Pool 1 material. HIC Pool 2 elution started at 124 mS/cm, and peaked at 84 mS/cm. Although baseline resolution was not obtained between the two peaks in this experiment, it was amongst the best resolutions observed to-date in a linear gradient.

Table 9 shows the 260 nm and 280 nm areas for each process fraction, the normalized 260 nm area (% A260) and the A260/A280 ratio. HIC Pool 1 had a larger 260 nm area than HIC Pool 2, corresponding to 59% of the normalized 260 nm area. The ratio of A260/A280 was 1.07 in both elution pools. This suggests that the separation in HIC chromatography did not enrich for DNA containing capsids and therefore was not dependent on the presence of DNA in the capsid.

TABLE 9
HIC Chromatogram Areas (mAU*mL) at 260 nm and 280 nm

| | A280 | A260 | % A260 | A260/A280 |
|---|---|---|---|---|
| FTW | 1019 | 1177 | 3% | 1.16 |
| HIC Pool 1 | 22308 | 23911 | 59% | 1.07 |
| HIC Pool 2 | 10837 | 11602 | 29% | 1.07 |
| DIW Strip | 1775 | 1774 | 4% | 1.00 |
| Low pH Strip | 2409 | 2198 | 5% | 0.91 |

### HIC Chromatography VG & Capsid Mass Balance

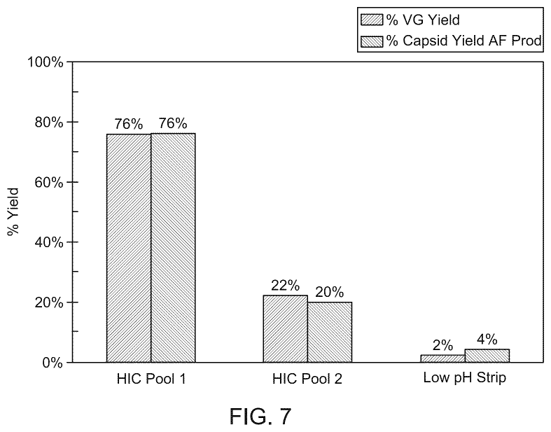

The volume pooled for HIC Pool 1 and HIC Pool 2 were 700 mL and 795 mL respectively. FIG. 7 shows the VG and capsid yield data based on AF Product titers normalized to mass balance. VG and capsid titers for the FTW fraction were below the limit of quantification and are not shown here. HIC Pool 1 contained most of the loaded material, comprising about 76% of capsid yield and 76% VG yield. This result corroborates the higher 260 nm area for Peak 1 observed in the chromatogram. The ratio of the VG titer per capsid titer was 19% for HIC Pool 1 and 22% for HIC Pool 2, suggesting this step did not enrich for DNA containing capsid.

### HIC Analytical Ultracentrifugation (AUC)

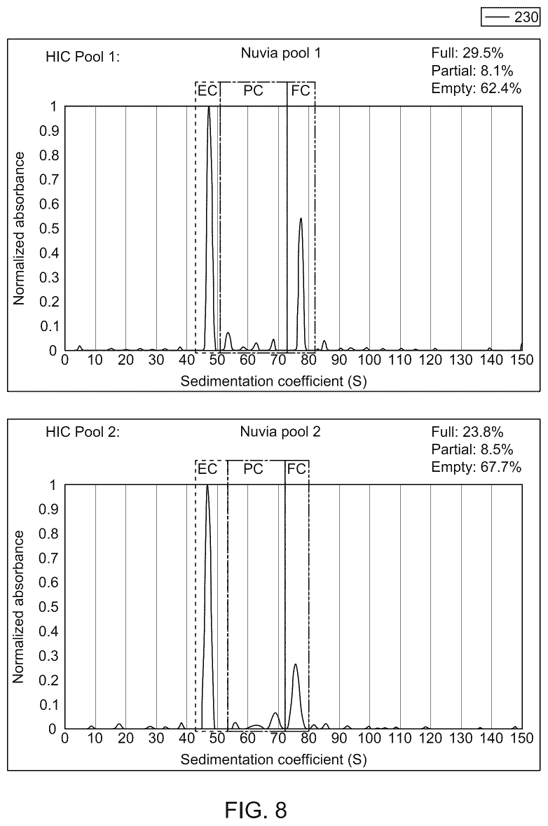

The AUC results for the Load, HIC Pool 1, HIC Pool 2 and Low pH Strip are shown in Table. FIG. 8 shows the AUC profiles of HIC Pool 1 and HIC Pool 2. The percentages of full and empty were similar among all the fractions, as well as the profiles for HIC Pool 1 and HIC Pool 2. These results demonstrate that the HIC chromatography is not enriching for DNA-containing capsids, but separating capsids based on surface differences.

TABLE 10
Summary of AUC Results for HIC Pool 1 and HIC Pool 2

| | AUC Empty % | AUC Partial % | AUC Full % |
|---|---|---|---|
| Nuvia Load | 63.9% | 6.6% | 29.5% |
| HIC Pool 1 | 62.4% | 8.1% | 29.5% |
| HIC Pool 2 | 67.7% | 8.5% | 23.8% |
| Low pH Strip | 59.6% | 15.6% | 24.8% |

### Tangential Flow Filtration

To concentrate and buffer exchange (BE) the IC Pool 1 and HIC Pool 2 materials, MicroKros 40 cm² 100K mPES membranes were used. HIC Pool 1 initial volume was 700 mL and had a capsid titer of 9.4×10¹² capsids/mL which resulted in a load challenge of 1.8×10¹⁸ capsids/m² of membrane. HIC Pool 2 initial volume was 795 mL and had a capsid titer of 2.2×10¹² capsids/mL, which resulted in a load challenge of 5.5×10¹⁷ capsids/m² of membrane. These load challenges were within the range of 5×10¹⁷ to 2×10¹⁸ capsids/m² targeted in TFF2. Each HIC pool was concentrated to target a capsid titer of 1×10¹⁴ capsids/mL.

Table 11 shows a summary of initial volumes and titers, as well as the titers obtained after TFF-2 and diafiltration.

TABLE 11
Titers Before and After TFF-2 and Diafiltration for HIC Pool 1 and Pool 2

| | | | | Target | | |
|---|---|---|---|---|---|---|
| | Initial | | Initial | BE | Titer after | Titer after |
| | Volume | Initial Titer | Titer | Volume | BE | BE |
| | (mL) | (Capsids/mL) | (VG/mL) | (mL) | (Capsids/mL) | (VG/mL) |
| HIC Pool 1 | 700 | 9.4 × 10¹² | 1.8 × 10¹² | 40 | 1.0 × 10¹⁴ | 2.2 × 10¹³ |
| HIC Pool 2 | 795 | 2.2 × 10¹² | 4.7 × 10¹¹ | 30 | 4.3 × 10¹³ | 7.2 × 10¹² |

During concentration, the permeate flow rate of the HIC Pool 1 was around 1 mL/min, and concentration of the pool took two days (560 minutes). After the first day the membrane was flushed, and a new membrane was used for the second day. Permeate flow rate of the HIC Pool 2 was around 3 mL/min and concentration was done in a single day (200 minutes).

After concentration, the solution was buffer exchanged with Neutralized Affinity Buffer (100 mM Glycine, 100 mM NaCl, pH 3.3, with 2% (v/v %) of 1M Tris, 100 mM NaCl, pH 10.9). Seven diafiltration volumes were planned for each buffer exchange step; however, after 193 mL and 234 mL for HIC Pool 1 and HIC Pool 2 respectively, the solution turned turbid. At that point, both buffer exchange processes were stopped. The buffer exchanged and concentrated pools were then filtered with a 25 mm EDF syringe filter. These samples were then named BE HIC Pool 1 and BE HIC Pool 2.

### AEX Chromatography

The buffer exchanged (BE) HIC Pool 1 and Pool 2 samples were further processed in the AEX process (described above) using a prepacked 10 mL Poros HQ50 resin. The estimated load challenge was 1.7×10¹⁴ capsids/mL resin for BE HIC Pool 1, and 1.1×10¹⁴ capsids/mL resin for BE HIC Pool 2.

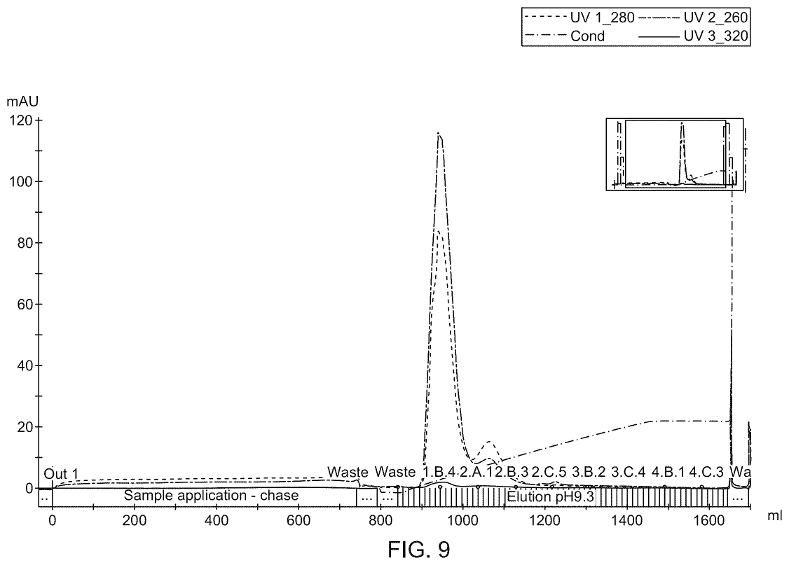

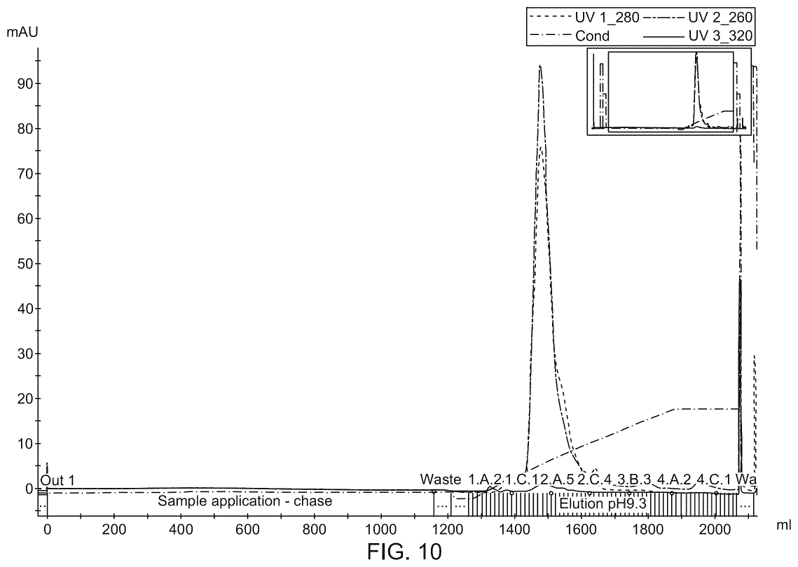

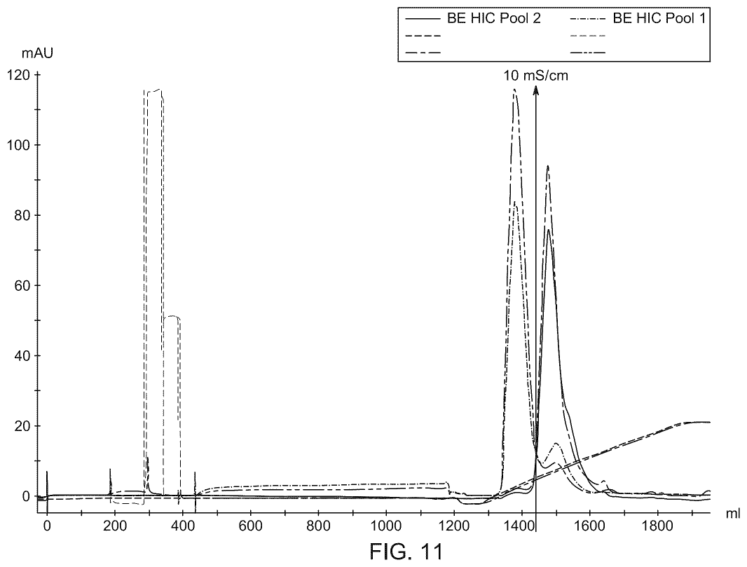

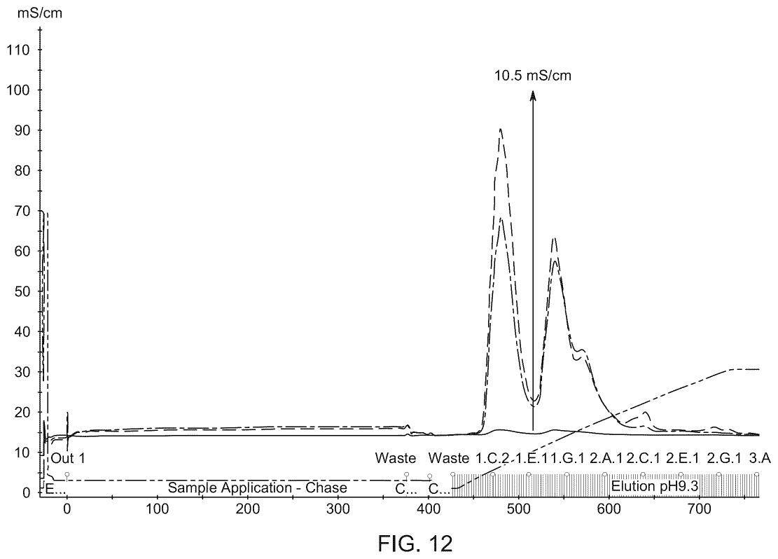

The chromatograms obtained for the AEX polishing step using BE HIC Pool 1 and BE HIC Pool 2 are shown in FIG. 9 and FIG. 10, respectively. FIG. 11 shows the overlay of the two AEX runs, and FIG. 12 shows a typical AEX chromatogram with gradient elution using AF Product material.

The chromatogram of the AEX step using BE HIC Pool 1 (FIG. 7) had the typical flowthrough profile and Peak 1 gradient elution. However, Peak 2 gradient elution was almost nonexistent. On the other hand, the chromatogram of the AEX step using BE HIC Pool 2 (FIG. 8) had no flowthrough, a small Peak 1 gradient elution, and a high Peak 2 gradient elution. The overlay of the AEX chromatograms of BE HIC Pool 1 and BE HIC Pool 2 (FIG. 11) resembles the typical AEX chromatogram (FIG. 12).

The 260 nm and 280 nm areas for each AEX process fractions are shown respectively in Table 12 and Table 13 for BE HIC Pool 1 and BE HIC Pool 2 load materials. For BE HIC Pool 1 load, the AEX Pool 1 had 73% of the normalized 260 nm area, while the FTW had 16% and the AEX Pool 2 only 10% (Table 12). The ratio of A260/A280 was 1.38 for HIC Pool 1, suggesting an enrichment of that pool for DNA containing capsids.

TABLE 12
BE HIC Pool 1 - AEX Chromatogram
Areas (mAU*mL) at 260 nm, 280 nm

| | A280 | A260 | % A260 | A260/A280 |
|---|---|---|---|---|
| FTW | 2264 | 1419 | 16% | 0.63 |
| AEX Pool 1 | 4613 | 6366 | 73% | 1.38 |
| AEX Pool 2 | 1246 | 863 | 10% | 0.69 |
| Strip | 83 | 103 | 1% | 1.25 |

For BE HIC Pool 2 load, the FTW UV absorbance was below the detection limit and the AEX Pool 1 had <5% of the normalized 260 nm area indicating low capsid concentration in those pools. The AEX Pool 2 had most of the 260 nm normalized area (92%), but low ratio of A260/A280 (1.08), suggesting that pool had low concentration of DNA containing capsids (Table 13).

TABLE 13
BE HIC Pool 2 - AEX Chromatogram
Areas (mAU*mL) at 260 nm, 280 nm

| | A280 | A260 | % A260 | A260/A280 |
|---|---|---|---|---|
| FTW | — | — | — | — |
| AEX Pool 1 | 143 | 233 | 4% | 1.62 |
| AEX Pool 2 | 5390 | 5832 | 92% | 1.08 |
| Strip | 179 | 256 | 4% | 1.43 |

### AEX Chromatography VG & Capsid Mass Balance

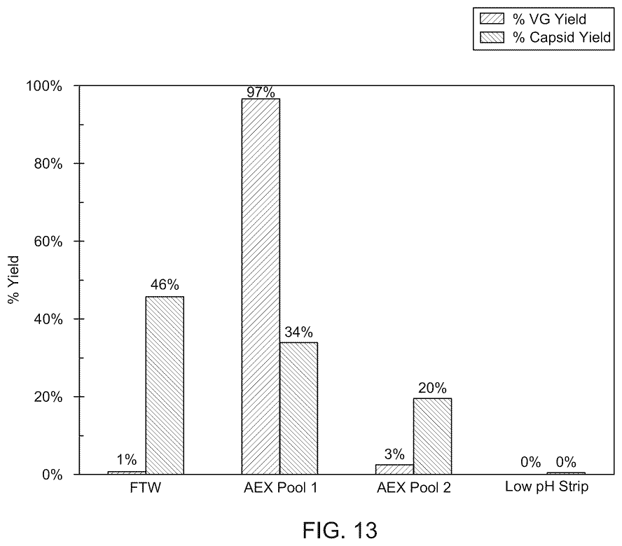

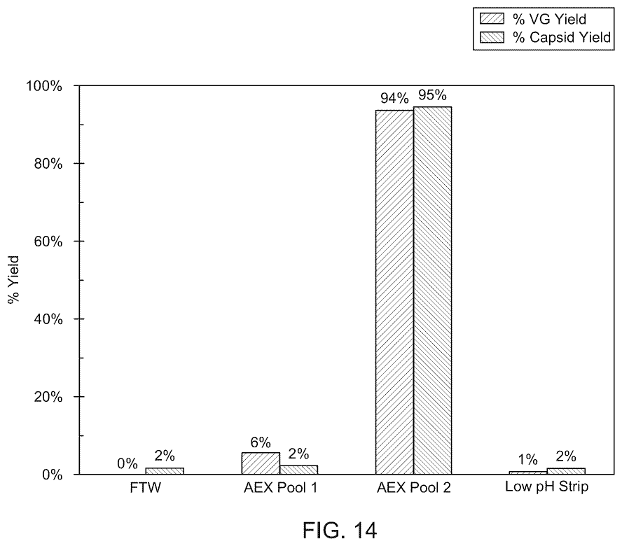

VG and capsid yield data of the AEX chromatography step with BE HIC Pool 1 and BE HIC Pool 2 load materials are shown in FIG. 13 and FIG. 14, respectively. Yields were calculated based on the titers of the BE HIC pools (because of the variability in the AEX load titers) and normalized to mass balance.

From the yield data, the BE HIC Pool 1 capsids partitioned between the AEX FTW, AEX Pool 1, and AEX Pool 2 fractions. However, the majority of the VGs in HIC Pool 1 were recovered in the AEX Pool 1, the Product pool. VG yield in the FTW and Pool 2 were <5%. These results are aligned with the 260 nm normalized areas for those pools and the high A260/A280 ratio for the AEX Pool 1 (Table 12).

From FIG. 14, the BE HIC Pool 2 material consisted mostly of capsids and VGs that were recovered in the AEX Non-Product Pool 2 fraction (>94%). VG and capsid yield in the AEX Pool 1 and FTW fractions were <6%. These results are aligned with the 260 nm normalized areas for those pools (Table 13).

### AEX Analytical Ultracentrifugation (AUC)

The AUC results for each fraction of the AEX chromatography step using BE HIC Pool 1 and BE HIC Pool 2 materials are shown in Table 14 and Table 15, respectively.

TABLE 14
BE HIC Pool 1 - Summary of AUC Results
for each Fraction of the AEX Step

| | AUC Empty % | AUC Partial % | AUC Full % |
|---|---|---|---|
| Load: BE HIC Pool 1 | 62.4% | 8.1% | 29.5% |
| AEX FTW | 96.6% | 3.3% | 0.1% |
| AEX Pool 1 | 5.1% | 7.1% | 87.7% |
| AEX Pool 2 | 87.9% | 9.1% | 3.0% |
| AEX Strip | 59.1% | 24.3% | 16.7% |

TABLE 15
BE HIC Pool 2 - Summary of AUC Results
for each Fraction of the AEX Step

| | AUC Empty % | AUC Partial % | AUC Full % |
|---|---|---|---|
| Load: BE HIC Pool 2 | 67.7% | 8.5% | 23.8% |
| AEX FTW | — | — | — |
| AEX Pool 1 | 18.8% | 8.3% | 72.9% |
| AEX Pool 2 | 63.2% | 8.1% | 28.7% |
| AEX Strip | 62.6% | 24.8% | 12.6% |

AUC results from the AEX chromatogram step using BE HIC Pool 1 showed that the AEX FTW and AEX Pool 2 mostly consisted of empty capsids (Table 14). The product peak (AEX Pool 1) consisted of 87.7% full capsids, similar to the AUC results of the HQ1 Product obtained in DEV-000169 (82.4% full).

With BE HIC Pool 2 material (Table 15), AUC results for the AEX Pool 1 (Product peak) showed a higher percentage of empty capsids (18.8%) and lower percentage of full (72.9%), suggesting this enrichment step was not as successful as for the BE HIC Pool 1. The AEX Pool 2 fraction had a higher percentage of full and was comparable to the load sample. AEX FTW of this process was not submitted for AUC due to low titers obtained.

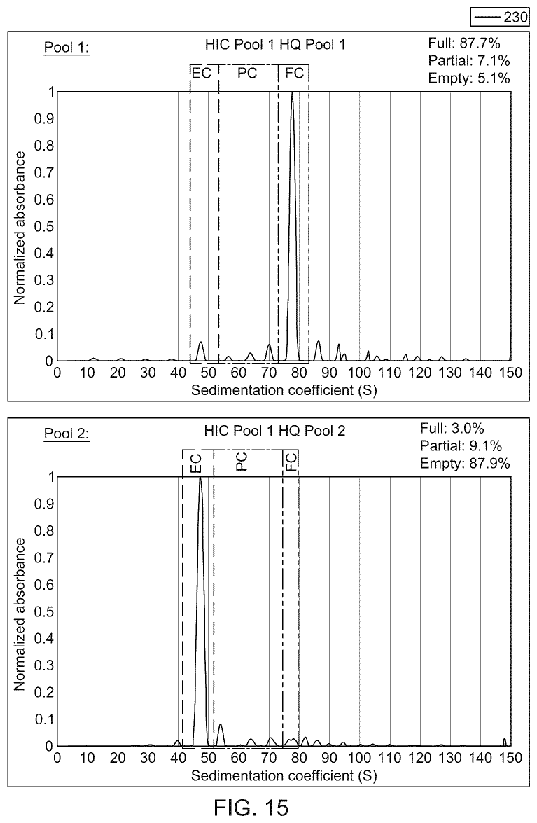

FIG. 15 shows the AUC profiles of AEX Pool 1 and AEX Pool 2 fractions of the AEX chromatography step using BE HIC Pool 1.

### Residuals, CE and SEC Results

Table 16 shows the capillary electrophoresis (CE) results for Pool 1 and Pool 2 of the 3 runs executed: HIC chromatography, AEX chromatography of BE HIC Pool 1, and AEX chromatography of BE HIC Pool 2. All pools were concentrated prior to testing. HIC Pool 2—AEX Pool 1 had too low concentration and was not submitted for CE.

TABLE 16
CE Results for the Runs Executed

| | | Total capsid | | | | |
|---|---|---|---|---|---|---|
| | | % | % | Area % | | |
| Sample Name | N | Purity | CV | VP1 | VP2 | VP3 |
| BE HIC Pool 1 | 3 | 99.29 | 1.05 | 10.64 | 12.94 | 75.69 |
| BE HIC Pool 2 | 3 | 99.71 | 0.50 | 10.79 | 9.42 | 79.52 |
| HIC Pool 1 - AEX Pool 1 | 3 | 99.66 | 0.60 | 8.52 | 10.20 | 80.93 |
| HIC Pool 1 - AEX Pool 2 | 3 | 99.80 | 0.35 | 9.35 | 10.68 | 79.77 |
| HIC Pool 2 - AEX Pool 1 | — | — | — | — | — | — |
| HIC Pool 2 - AEX Pool 2 | 3 | 99.77 | 0.40 | 10.24 | 10.44 | 79.09 |

The percentage purity of all samples was above 99%. BE HIC Pool 1 and HIC Pool 1—AEX Pool 1 electropherograms had 3 peaks at around 24 min retention, typical of DNA. These are the pools of interest (product pools of each process). HIC Pool 1—AEX Pool 1 had the highest DNA peaks and the highest AUC percentage full. The other electropherograms had very small to non-existent DNA peaks.

The area percentage of VP1, VP2 and VP3 varied among the samples. For the HIC chromatography samples, the more hydrophobic BE HIC Pool 2 had lower VP2 area and higher VP3 area than the less hydrophobic BE HIC Pool 1 material (Table 16). It is still unclear if this change is related to the increase in hydrophobicity of the capsids. VP2 peak maximum in the HIC Pool 2—AEX Pool 2 was also lower than VP1 peak maximum, but the VP2 area of that sample was similar to the other AEX elution pools (Area % VP2˜10.4%).

Size Exclusion Chromatography High-Performance Liquid Chromatography (SEC-HPLC) results are shown in Table 17. SEC samples were not concentrated prior to analysis except for BE HIC Pool 1 and BE HIC Pool 2 which were the concentrated, buffer exchanged samples.

TABLE 17
SEC Results for the Runs Executed

| | | | Estimate | Titer |
|---|---|---|---|---|
| | | % | Titer | ELISA |
| Sample Name | N | Aggregate | (caps/mL) | (caps/mL) |
| HIC Pool 1 | 2 | 4.05 | 1.51 × 10¹³ | 9.37 × 10¹² |
| HIC Pool 2 | 2 | 52.72 | Low Conc. | 2.17 × 10¹² |
| BE HIC Pool 1 | 2 | 1.72 | 1.54 × 10¹⁴ | 1.04 × 10¹⁴ |
| BE HIC Pool 2 | 2 | 6.02 | 2.48 × 10¹³ | 4.26 × 10¹³ |
| HIC Pool 1 - AEX Pool 1 | 2 | 0.40 | 1.11 × 10¹³ | 4.75 × 10¹² |
| HIC Pool 1 - AEX Pool 2 | 2 | 7.13 | Low Conc. | 1.46 × 10¹² |
| HIC Pool 2 - AEX Pool 1 | 2 | 30.25 | Low Conc. | 1.54 × 10¹¹ |
| HIC Pool 2 - AEX Pool 2 | 2 | 17.68 | Low Conc. | 3.48 × 10¹² |

In general, samples with capsid titer below 4×10¹² capsids/mL had high % Aggregate. These samples had low absorbance signal in the monomer peak and high noise, which increased % Aggregate. HIC Pool 1 and HIC Pool 2 were the original fractions from the HIC step, while the BE HIC Pool 1 and BE HIC Pool 2 were the samples from the HIC step after concentration, buffer exchange and EDF filtration. For both pools, % Aggregate decreased after TFF-2. The lowest % Aggregate was obtained for the product peak HIC Pool 1—AEX Pool 1 (0.40%). Table 18 shows the hcDNA and HCP residuals for the two main product peaks of the process. Table 19 shows the plasmid residuals for the main product peaks of the process. The concentrations of all process residuals were low for both BE HIC Pool 1 and HIC Pool 1—AEX Pool 1.

TABLE 18
hcDNA and HCP Residuals for the Main Product Pools

| | hcDNA | ng hcDNA/ | HCP ELISA | ng HCP/1 × 10¹³ |
|---|---|---|---|---|
| Sample Name | (ng/mL) | 1 × 10¹² VG | (ng/mL) | VGs |
| BE HIC Pool 1 | 155.8 | 7.2 × 10¹ | BLOQ | — |
| HIC Pool 1 - | 13.6 | 3.5 × 10¹ | BLOQ | — |
| AEX Pool 1 | | | | |

TABLE 19
Plasmids Residuals for the Main Product Pools

| | Rep/Cap | Rep/Cap/ | E1A | E1A/ | pHelper | pHelper/ |
|---|---|---|---|---|---|---|
| Sample Name | (/mL) | 1 × 10¹³ VG | (/mL) | 1 × 10¹³ VG | (/mL) | 1 × 10¹³ VG |
| BE HIC Pool 1 | 3.1 × 10⁹ | 1.4 × 10⁹ | BLOQ | — | 3.1 × 10⁹ | 1.4 × 10⁹ |
| HIC Pool 1 - | 3.6 × 10⁹ | 9.3 × 10⁹ | BLOQ | — | 5.3 × 10⁸ | 1.4 × 10⁹ |
| AEX Pool 1 | | | | | | |

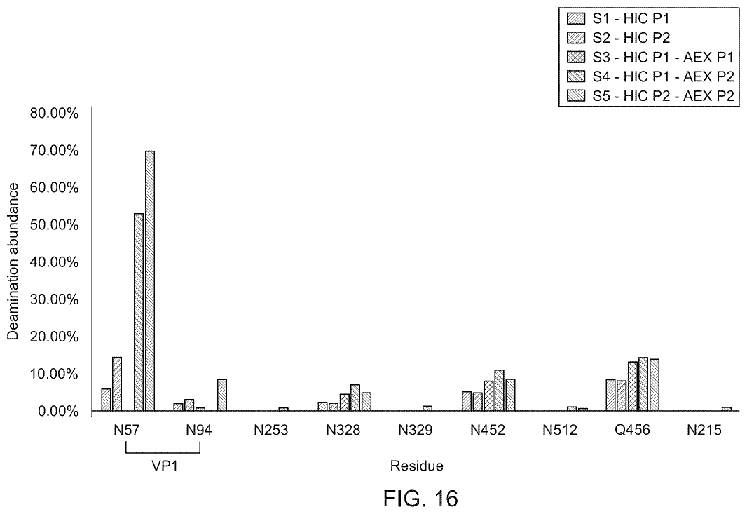

According to the experiments described in this Example, HIC was performed first to isolate HIC Pool 1 (least hydrophobic material) and then AEX was performed to isolate AEX Pool 1 (least charged material). As shown in FIG. 16, the target product resulted in 0% N57 deamidation.

### Example 4: Identification and Control of AAV Charge Heterogeneity Through Optimized Bioreactor Operation

Small charge differences in recombinant adeno-associated virus (rAAV) capsids are traditionally exploited with anion exchange (AEX) chromatography to separate empty and full capsids. During the development of this step for rAAV9, it was noted that capsids are not uniform in charge and can be found in various AEX fractions eluted at variable salt concentrations. The observed broad distribution of capsids suggested a heterogenous charge profile. To understand the root of this heterogeneity, the production time in the bioreactor was manipulated to determine where in the system the heterogeneity is greatest (intracellular or extracellular).

HEK293 cells were transfected using a transient transfection platform and incubated for a duration of either 3 or 6 days before harvest. In one arm, a detergent lysis was performed at day 3 post-transfection and the full lysate was collected. For the other arm, the cell and supernatant were separated by centrifugation at days 3 and 6 post-transfection. The cell pellets were resuspended and lysed to extract intracellular capsids. After harvest, all feed-streams were purified using affinity chromatography followed by an AEX purification step to enrich for full capsids and examine the observed charge distribution.

Empty capsids had more heterogeneous charge distributions than DNA-containing capsids, and, surprisingly, the intracellular capsids were more negatively charged than extracellular capsids. Further investigation revealed that extracellular capsids were composed of less empty capsids, which helped to explain their more uniform charge distribution. LC-MS/MS of the various feed-streams unexpectedly showed that intracellular capsids had more asparagine N57 deamidation right out of the bioreactor than extracellular capsids and increased levels from day 3 to day 6 post-transfection. A linear correlation exists between N57 deamidation and capsids lost to the AEX strip, demonstrating the N57 deamidation was responsible for the increase in charge heterogeneity observed through AEX mass balance. Deamidation at N57 in the product was reduced by ˜2× through AEX for all feed-streams tested, however, that step was not sufficient in completely removing deamidated species. Potency, as measured by GFP expression and fluorescence, significantly dropped from day 3 to day 6 post-transfection for all feed-streams.

Thus, deamidation at N57 occurs within the bioreactor and is responsible for charge heterogeneity witnessed during the AEX step. While AEX is capable of removing some deamidated species generated through the bioreactor process, harvest time is a more significant parameter that can be controlled to decrease N57 deamidation and increase the potency of rAAV products. Finally, scale-up data demonstrated that it is possible to further reduce N57 deamidation and increase potency without significantly sacrificing titer by shortening the bioreactor duration to two days post-transfection.

### Example 5: Comparison of Day 2 Versus Day 3 Upstream Processing at Different Post-Transfection Temperatures

To determine the effect of upstream processes on production of functional capsids, AAV9 viruses were transfected, and post-transfection temperature was maintained at 37° C. or shifted to 39° C. prior to harvest. Cells were harvested at 2 days and 3 days post transfection from bioreactors at each temperature. Transfected cells were then lysed and purified. Purification included affinity chromatography followed by an AEX purification step to further enrich for full capsids.

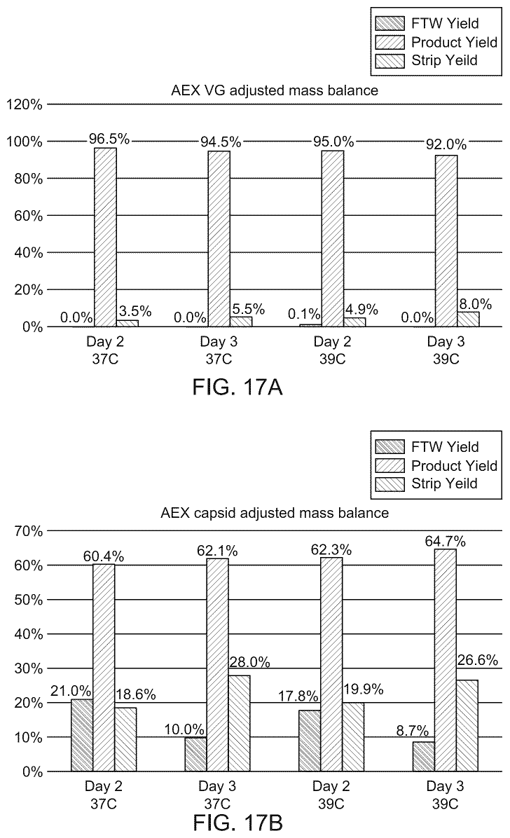

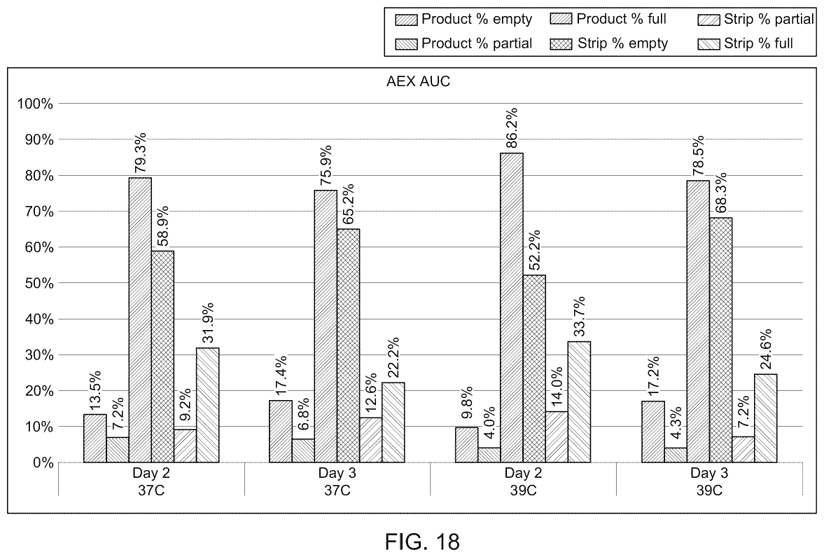

Following AEX purification, VG and capsid yields were assessed from FTW, product, and strip fractions. See FIGS. 17A and 17B. From day 2 to day 3, a slight decrease in product yield was observed at both 37° C. or 39° C. Closer inspection revealed that this was likely due to an increase in VG found in the strip fraction. Interestingly, VG titers showed a slight increase in loss to strip on Day 3 versus Day 2 for both temperatures indicating that the feed-stream is more negatively charged. With respect to capsid yield, a decrease in FTW loss on Day 3 versus Day 2 and an increase in capsid loss to strip on Day 3 versus Day 2 was observed. These findings also support the feed-stream as having more negative charge. Investigation into the AUCs for day 2 and day 3 harvest showed a higher percentage of empty capsids in the product peak and full capsids in the non-product peak from the strip on day 3 as compared to day 2, further supporting an increase in negative charge of the feed-stream. See FIG. 18.

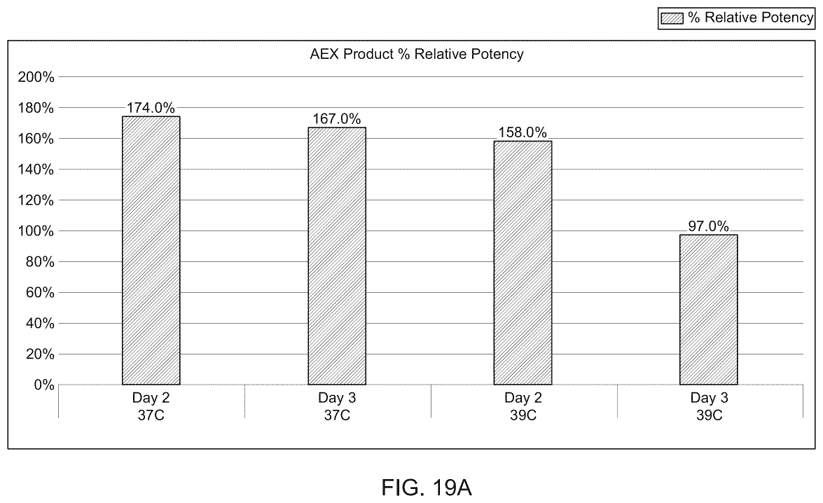

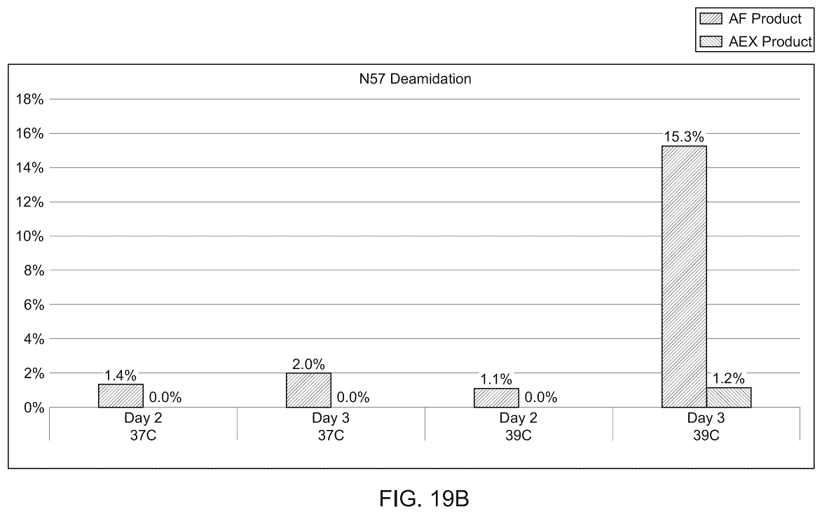

Given that potency and percent deamidation are highly correlated, both parameters were examined after AEX purification. Capsids harvested on 2 days post transfection had higher potency compared to day 3; furthermore, when post transfection temperature was set to 37° C., those capsids displayed the highest overall relative potency (FIG. 19A). Notably, the percent of N57 deamidation detected correlated with potency. As deamidation increased, potency decreased. This was especially notable for the cells harvested on day 3 at 39° C. as these cells had significantly lower potency and the highest percentage of N57 deamidation. See FIG. 19B.

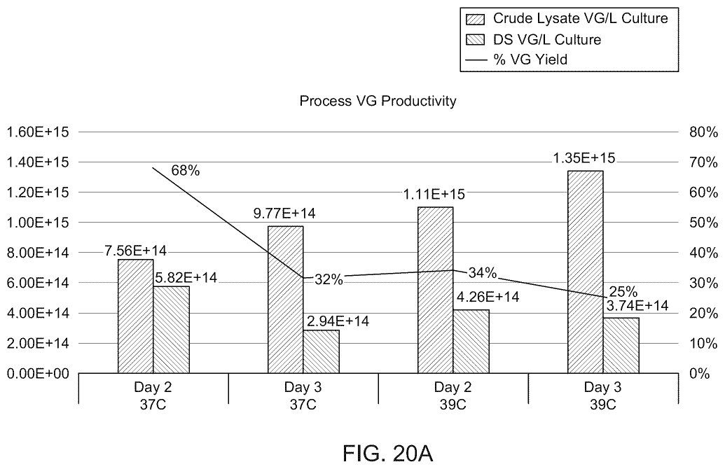

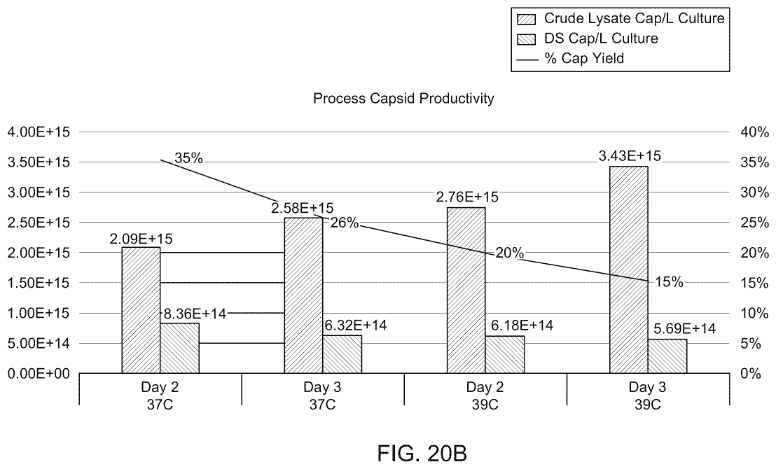

Investigation into VG and capsid productivity over the total upstream process revealed that both VG and capsid productivity increased from day 2 to day 3; however total recovery over the course of the upstream process decreased. This was observed at both post transfection temperatures. See FIGS. 20A and 20B.

1. An adenovirus-associated virus (AAV) product comprising functional capsids containing a transgene, wherein less than 5% of the capsids in the AAV product have a deamidated N57 on VP1.

2-4. (canceled)

5. The AAV product of claim 1, wherein less than 1% of the capsids in the AAV product have a deamidated N57 on VP1.

6. The AAV product of claim 1, wherein the AAV product comprises less than 10% non-functional capsids.

7. (canceled)

8. (canceled)

9. The AAV product of claim 1, wherein deamidation is detected by liquid chromatography-mass spectrometry.

10-19. (canceled)

20. A method of producing the adenovirus-associated virus (AAV) product of claim 1 from a composition comprising AAV capsids, the method comprising separating non-functional capsids in the composition from functional capsids containing a transgene, wherein the separating comprises:
(a) hydrophobic interaction chromatography (HIC),
(b) anion-exchange chromatography with a fatty acid mobile phase, or
(c) mixed-mode anion-exchange chromatography.

21. The method of claim 20, wherein the separating further comprises anion-exchange chromatography (AEX).

22-24. (canceled)

25. The method of claim 20, wherein the separating comprises HIC.

26. The method of claim 25, wherein the separating further comprises AEX.

27. The method of claim 26, wherein AEX is performed without a fatty acid mobile phase.

28. The method of claim 26, wherein HIC precedes AEX.

29. The method of claim 20, wherein the separating comprises anion-exchange chromatography with a fatty acid mobile phase.

30. The method of claim 29, wherein the fatty acid mobile phase comprises octanoic or heptanoic acid.

31. The method of claim 20, wherein the separating comprises mixed-mode resin chromatography.

32. The method of claim 31, wherein the mixed mode resin is a hydrophobic anion exchange resin.

33. The method of claim 20, further comprising transfecting target cells in a bioreactor to produce unseparated capsids, wherein the unseparated capsids are maintained in the bioreactor for up to two days after transfection of the target cells.

34. (canceled)

35. A method of treating a subject in need of a protein produced by a transgene comprising administering to the subject the AAV product of claim 1.

36. The method of claim 20, wherein the AAV product has greater transgene expression in a host cell than a control AAV product purified by a method consisting of (i) ultracentrifugation or (ii) AEX without fatty acid mobile phase.

37. The method of claim 20, wherein the AAV product has a lower ratio of VP1 to VP2 than a control AAV product purified by a method consisting of (i) ultracentrifugation or (ii) AEX without fatty acid mobile phase.

38. The method of claim 33, wherein AAV capsids are harvested at 2 days post-transfection.

39. The method of claim 33, wherein post-transfection temperature in the bioreactor is 37° C.
